(19) United States
(12) Patent Application Publication
(10) Pub. No.: US 2012/0027697 A1
(43) Pub. Date: Feb. 2, 2012

(54) PLANT EXTRACTS, COMPOSITIONS CONTAINING SAME, AND USES THEREOF

(76) Inventor: Namita Deo, Newark, NJ (US)

(21) Appl. No.: 13/017,179

(22) Filed: Jan. 31, 2011

Related U.S. Application Data
(63) Continuation-in-part of application No. 12/729,718, filed on Mar. 23, 2010.

(51) Int. Cl. A61K 8/97; A61K 36/23; A61K 36/47; A61K 36/75; A61K 36/185; A61K 36/28; A61Q 17/04; A61Q 19/04; A61Q 19/08; A61P 17/00; A61P 29/00; A61P 5/00; A61P 39/06; A61P 31/00; A61P 31/10; A61Q 5/06; A61Q 11/00; C11D 3/382; C09K 3/00; C08K 5/00; C09K 8/584; C10M 169/04; C10L 1/10; A23L 1/212; A61K 36/68

(52) U.S. Cl. 424/58; 424/725; 424/769; 424/59; 424/70.6; 510/108; 510/344; 510/365; 510/109; 510/218; 252/380; 252/182.12; 516/10; 106/400; 507/203; 508/110; 44/307; 426/615

(57) ABSTRACT

This disclosure relates to plant extracts selected from Bacopa plant extracts, Centella plant extracts, Jatropha plant extracts, Aegle plant extracts, Terminalia plant extracts, Phyllanthus plant extracts, Spilanthes plant extracts, and mixtures thereof; compositions containing plant extracts selected from Bacopa plant extracts, Centella plant extracts, Jatropha plant extracts, Aegle plant extracts, Terminalia plant extracts, Phyllanthus plant extracts, Spilanthes plant extracts, and mixtures thereof; and to the use of these compositions in a variety of applications including, for example, cosmetic and/or pharmaceutical preparations, therapeutic preparations, and personal care preparations. A preferred Bacopa plant extract is obtained from Bacopa monniera, a preferred Centella plant extract is obtained from Centella asiatica, a preferred Jatropha plant extract is obtained from Jatropha curcas, a preferred Aegle plant extract is obtained from Aegle marmelos, a preferred Terminalia plant extract is obtained from Terminalia chebula or Terminalia bellerica, a preferred Phyllanthus plant extract is obtained from Phyllanthus emblica (Emblica officinalis), and a preferred Spilanthes plant extract is obtained from Spilanthes acmella (Acmella oleracea).

This application is a continuation-in-part of U.S. patent application Ser. No. 12/729,718, filed Mar. 23, 2010, which is incorporated herein by reference in its entirety.

### BACKGROUND

1. Field of the Disclosure

This disclosure relates to plant extracts selected from Bacopa plant extracts, Centella plant extracts, Jatropha plant extracts, Aegle plant extracts, Terminalia plant extracts, Phyllanthus plant extracts, Spilanthes plant extracts, and mixtures thereof; compositions containing plant extracts selected from Bacopa plant extracts, Centella plant extracts, Jatropha plant extracts, Aegle plant extracts, Terminalia plant extracts, Phyllanthus plant extracts, Spilanthes plant extracts, and mixtures thereof; and to the use of these compositions in a variety of applications including, for example, cosmetic and/or pharmaceutical preparations, therapeutic preparations, and personal care preparations.

2. Discussion of the Background Art

For many years, plant extracts have been used for medicinal and also for cosmetic purposes in various cultures. New plants are being extracted and the extracts studied for their cosmetic and pharmaceutical effects in the search to find further plants with a new or different action spectrum. Many plants whose value was not known and which were regarded as exotic and unimportant are now used in the cosmetic and pharmaceutical fields.

Today, cosmeceutical preparations are available to the consumer in a variety of combinations. Consumers not only expect these cosmeceuticals to have a certain care effect or to eliminate a certain deficiency, they are also increasingly demanding products which combine several properties and thus show an improved performance spectrum. There is a particular interest in substances which both positively influence the technical properties of the cosmeceutical product, such as storage stability, light stability and formulatability, and at the same time represent active principles that impart, for example, caring, moisturizing, irritation-inhibiting, inflammation-inhibiting and/or sun protection properties to the skin/hair. In addition, consumers demand high dermatological compatibility and preferably the use of natural products.

There is also a general demand for cosmeceutical preparations that, by virtue of their special composition, have high-quality technical properties and which, in addition, are distinguished by additional properties for the skin/hair. These cosmeceutical preparations should not only be beneficial to consumers, but also should be environmentally friendly, e.g., not a source of synthetic chemical pollution.

The present disclosure provides many advantages, which shall become apparent as described below.

### SUMMARY OF THE DISCLOSURE

This disclosure relates in part to cosmeceutically active extracts selected from the group consisting of Bacopa plant extracts, Centella plant extracts, Jatropha plant extracts, Aegle plant extracts, Terminalia plant extracts, Phyllanthus plant extracts, Spilanthes plant extracts, and mixtures thereof.

This disclosure also relates in part to cosmeceutically active extracts selected from the group consisting of a sunless tanning active extract of a Bacopa plant, a Centella plant, a Jatropha plant, an Aegle plant, a Terminalia plant, a Phyllanthus plant, a Spilanthes plant, or mixtures thereof; a sunscreen active extract of a Bacopa plant, a Centella plant, a Jatropha plant, an Aegle plant, a Terminalia plant, a Phyllanthus plant, a Spilanthes plant, or mixtures thereof; an ultraviolet radiation protective extract of a Bacopa plant, a Centella plant, a Jatropha plant, an Aegle plant, a Terminalia plant, a Phyllanthus plant, a Spilanthes plant, or mixtures thereof; an anti-aging active extract of a Bacopa plant, a Centella plant, a Jatropha plant, an Aegle plant, a Terminalia plant, a Phyllanthus plant, a Spilanthes plant, or mixtures thereof; an anti-wrinkle active extract of a Bacopa plant, a Centella plant, a Jatropha plant, an Aegle plant, a Terminalia plant, a Phyllanthus plant, a Spilanthes plant, or mixtures thereof; a moisturizing active extract of a Bacopa plant, a Centella plant, a Jatropha plant, an Aegle plant, a Terminalia plant, a Phyllanthus plant, a Spilanthes plant, or mixtures thereof; a skin soothing active extract of a Bacopa plant, a Centella plant, a Jatropha plant, an Aegle plant, a Terminalia plant, a Phyllanthus plant, a Spilanthes plant, or mixtures thereof; a skin/hair softening active extract of a Bacopa plant, a Centella plant, a Jatropha plant, an Aegle plant, a Terminalia plant, a Phyllanthus plant, a Spilanthes plant, or mixtures thereof; a skin/hair treatment active extract of a Bacopa plant, a Centella plant, a Jatropha plant, an Aegle plant, a Terminalia plant, a Phyllanthus plant, a Spilanthes plant, or mixtures thereof; an anti-inflammatory active extract of a Bacopa plant, a Centella plant, a Jatropha plant, an Aegle plant, a Terminalia plant, a Phyllanthus plant, a Spilanthes plant, or mixtures thereof; a cosmeceutical preservative active extract of a Bacopa plant, a Centella plant, a Jatropha plant, an Aegle plant, a Terminalia plant, a Phyllanthus plant, a Spilanthes plant, or mixtures thereof; a cosmeceutical stabilizer active extract of a Bacopa plant, a Centella plant, a Jatropha plant, an Aegle plant, a Terminalia plant, a Phyllanthus plant, a Spilanthes plant, or mixtures thereof; an antioxidant active extract of a Bacopa plant, a Centella plant, a Jatropha plant, an Aegle plant, a Terminalia plant, a Phyllanthus plant, a Spilanthes plant, or mixtures thereof; a free radical inhibitive extract of a Bacopa plant, a Centella plant, a Jatropha plant, an Aegle plant, a Terminalia plant, a Phyllanthus plant, a Spilanthes plant, or mixtures thereof; a cleansing active extract of a Bacopa plant, a Centella plant, a Jatropha plant, an Aegle plant, a Terminalia plant, a Phyllanthus plant, a Spilanthes plant, or mixtures thereof; an anti-microbial active extract of a Bacopa plant, a Centella plant, a Jatropha plant, an Aegle plant, a Terminalia plant, a Phyllanthus plant, a Spilanthes plant, or mixtures thereof; anti-fungal active extract of a Bacopa plant, a Centella plant, a Jatropha plant, an Aegle plant, a Terminalia plant, a Phyllanthus plant, a Spilanthes plant, or mixtures thereof; a hair coloring active extract of a Bacopa plant, a Centella plant, a Jatropha plant, an Aegle plant, a Terminalia plant, a Phyllanthus plant, a Spilanthes plant, or mixtures thereof; and an oral cleaning/deodoring active extract of a Bacopa plant, a Centella plant, a Jatropha plant, an Aegle plant, a Terminalia plant, a Phyllanthus plant, a Spilanthes plant, or mixtures thereof.

This disclosure further relates in part to cosmeceutical compositions that contain, in an effective concentration, at least one of a Bacopa plant extract, a Centella plant extract, a Jatropha plant extract, an Aegle plant extract, a Terminalia plant extract, a Phyllanthus plant extract, a Spilanthes plant extract, or mixtures thereof, and at least one cosmeceutically acceptable carrier.

This disclosure yet further relates in part to a method of protecting or treating skin and/or hair of humans which comprises applying to the skin and/or hair a cosmeceutical composition comprising, in an effective concentration, at least one of a Bacopa plant extract, a Centella plant extract, a Jatropha plant extract, an Aegle plant extract, a Terminalia plant extract, a Phyllanthus plant extract, a Spilanthes plant extract, or mixtures thereof, and at least one cosmeceutically acceptable carrier.

This disclosure also relates in part to extracts selected from the group consisting of Bacopa plant extracts, Centella plant extracts, Jatropha plant extracts, Aegle plant extracts, Terminalia plant extracts, Phyllanthus plant extracts, Spilanthes plant extracts, and mixtures thereof.

This disclosure further relates in part to extracts selected from the group consisting of a stabilizer active extract of a Bacopa plant, a Centella plant, a Jatropha plant, an Aegle plant, a Terminalia plant, a Phyllanthus plant, a Spilanthes plant, or mixtures thereof; a surfactant active extract a Bacopa plant, a Centella plant, a Jatropha plant, an Aegle plant, a Terminalia plant, a Phyllanthus plant, a Spilanthes plant, or mixtures thereof; a preservative active extract a Bacopa plant, a Centella plant, a Jatropha plant, an Aegle plant, a Terminalia plant, a Phyllanthus plant, a Spilanthes plant, or mixtures thereof; a treatment active extract a Bacopa plant, a Centella plant, a Jatropha plant, an Aegle plant, a Terminalia plant, a Phyllanthus plant, a Spilanthes plant, or mixtures thereof; a dispersant active extract of a Bacopa plant, a Centella plant, a Jatropha plant, an Aegle plant, a Terminalia plant, a Phyllanthus plant, a Spilanthes plant, or mixtures thereof; a chelating active extract of a Bacopa plant, a Centella plant, a Jatropha plant, an Aegle plant, a Terminalia plant, a Phyllanthus plant, a Spilanthes plant, or mixtures thereof; an emulsifier active extract of a Bacopa plant, a Centella plant, a Jatropha plant, an Aegle plant, a Terminalia plant, a Phyllanthus plant, a Spilanthes plant, or mixtures thereof; a surface modifying active extract of a Bacopa plant, a Centella plant, a Jatropha plant, an Aegle plant, a Terminalia plant, a Phyllanthus plant, a Spilanthes plant, or mixtures thereof; a foaming/frothing active extract of a Bacopa plant, a Centella plant, a Jatropha plant, an Aegle plant, a Terminalia plant, a Phyllanthus plant, a Spilanthes plant, or mixtures thereof; a surface active agent active extract of a Bacopa plant, a Centella plant, a Jatropha plant, an Aegle plant, a Terminalia plant, a Phyllanthus plant, a Spilanthes plant, or mixtures thereof; a cleansing/degreasing active extract of a Bacopa plant, a Centella plant, a Jatropha plant, an Aegle plant, a Terminalia plant, a Phyllanthus plant, a Spilanthes plant, or mixtures thereof; and a rheology modifying active extract of a Bacopa plant, a Centella plant, a Jatropha plant, an Aegle plant, a Terminalia plant, a Phyllanthus plant, a Spilanthes plant, or mixtures thereof.

This disclosure yet further relates in part to compositions that contain at least one of a Bacopa plant extract, a Centella plant extract, a Jatropha plant extract, an Aegle plant extract, a Terminalia plant extract, a Phyllanthus plant extract, a Spilanthes plant, or mixtures thereof.

This disclosure also relates to a method comprising: (i) providing a substrate or medium; (ii) providing a composition comprising at least one of a Bacopa plant extract, a Centella plant extract, a Jatropha plant extract, an Aegle plant extract, a Terminalia plant extract, a Phyllanthus plant extract, a Spilanthes plant, or mixtures thereof; and (iii) contacting the substrate or medium with the composition.

This disclosure further relates in part to a method comprising: (i) providing a substrate or medium, (ii) providing a composition comprising at least one of a Bacopa plant extract, a Centella plant extract, a Jatropha plant extract, an Aegle plant extract, a Terminalia plant extract, a Phyllanthus plant extract, a Spilanthes plant, or mixtures thereof, and (iii) contacting the substrate or medium with the composition.

Further objects, features and advantages of the present disclosure will be understood by reference to the following drawings and detailed description.

### BRIEF DESCRIPTION OF THE DRAWINGS

FIG. 1 graphically depicts the UV visible spectra of Bacopa monniera extract at different concentrations. As shown in FIG. 1, the Bacopa monniera extract can absorb light in the entire UV range from 190-400 nm.

FIG. 2 graphically depicts the free radical scavenging activity of Bacopa monniera extract by electron spin resonance (ESR). As shown in FIG. 2, 100 μg of Bacopa monniera extract can scavenge completely 1 mg of nitroxide free radical tetramethylpiperidinyl-1-oxy (Tempo).

### DETAILED DESCRIPTION OF THE DISCLOSURE

As used herein, an “effective concentration” or “effective amount” means a quantity of the composition provided for administration and at a particular dosing regimen, that is sufficient to achieve a desired appearance, e.g., sunless tanning, therapeutic, e.g., moisturizer, and/or prophylactic effect, e.g., sunscreen. For example, an effective amount may result in a desired sunless tan or the prevention of sunburn or a decrease in the symptoms associated with a condition that is being treated. The amount of the composition administered to the subject will depend on the type and severity of the condition, and the characteristics of the subject, where such characteristics include general health, age, sex, body weight and tolerance to the active agents in the compositions. Those skilled in the art will be able to determine appropriate dosages depending on these and other factors. The compositions of this disclosure can also be administered in combination with one or more additional cosmetic, therapeutic or prophylactic compounds. As used herein, the term “subject” is preferably a mammal, such as a human.

This disclosure addresses the problem of providing cosmetic and/or pharmaceutical preparations that would meet the requirements for cosmetic formulations, such as storage stability and dermatological compatibility, and in addition would have improved moisture-regulating, caring and protecting properties for human skin/hair. This disclosure uses plant extracts from plants which had not hitherto been known for cosmeceutical applications and to make their ingredients suitable for use as active principles in cosmetic and/or pharmaceutical preparations. As used herein, “cosmeceutical(s)” refers to cosmetic and/or pharmaceutical preparations. For purposes of this disclosure, cosmeceutical(s) include, but are not limited to, therapeutic and personal care preparations.

Bacopa is a genus containing from about 70 to about 100 aquatic plants. Bacopa is a member of the Plantaginaceae family. It is commonly known as Waterhyssop. Bacopa plants are annual or perennial, decumbent or erect stemmed plants. The leaves are opposite or whorled, and sessile. The leaf blade is regular, round to linear, and the venation is palmate or pinnate. The stems are hairy or smooth. The flowers are produced solitary or in pairs from the leaf axil. The flowers are usually radially symmetrical having 5 sepals and 5 petals, and are usually white, blue or purple in color. Dispersal and propagation of Bacopa plants is by seeds and stem fragments.

Bacopa plants are found in tropical and subtropical regions of the world. For example, Bacopa monniera commonly grows in marshy areas throughout India, Nepal, Sri Lanka, China, Taiwan, and Vietnam. Bacopa plants are also found in Florida and other southern states of the U.S.A. where they can be grown in damp conditions by a pond or bog garden.

Illustrative species include, for example, Bacopa australis, Bacopa caroliniana (e.g., lemon bacopa, blue water hyssop and giant bacopa), Bacopa crenata, Bacopa eisenii, Bacopa madagascariensis, Bacopa monniera (e.g., water hyssop, moneywort and herb of grace), Bacopa myriophylloides, Bacopa rotundifolia, and the like. Bacopa extracts may be prepared using methods known to those skilled in the art. Bacopa extracts may be present in the compositions of this disclosure at a concentration of at least about 0.001%, preferably at least about 1%, and more preferably at least about 5%. The concentration of Bacopa extracts in the compositions of this disclosure typically is not greater than about 20% or about 30%, and usually is not greater than about 10% or about 15%.

The extracts of the genus Bacopa according to this disclosure generally contain ingredients from the group consisting of alkaloids (e.g., brahmine and herpestine), saponins (e.g., d-mannitol and hersaponin, acid A and monnierin), and flavonoids (e.g., luteolin and apigenin). Other extracts include betulic acid, stigmastarol, β-sitosterol, bacosides and bacopasaponins. Illustrative of such bacosides and bacopasaponins include, for example, bacosides A, bacosides B, bacopaside II, bacopaside I, bacopaside X, bacopasaponin C, bacopaside N2, bacopasaponin F, bacopasaponin E, bacopaside N1, bacopaside III, bacopaside IV, and bacopaside V. These ingredients differ in composition according to the starting material and extraction method selected.

Mixtures of Bacopa plant extracts may also be useful in this disclosure. Mixtures of ingredients, e.g., individual molecular compositions, from one or more Bacopa plant extracts may further be useful in this disclosure. As used herein, “extract(s)” are plant extracts including individual molecular compositions that can be separated and isolated from the plant extracts.

Centella is a member of the Mackinlayaceae family. It is commonly known as Gotu Kola. Centella plants are slender stems and creeping stolons. They are green to reddish green in color and interconnecting one plant to another. Centella plants have long-stalked, green, reniform leaves with rounded apices which have a smooth texture with palmately netted veins. The rootstock consists of rhizomes that are creamish in color and covered with root hairs. The flowers are pinkish to red in color. Each flower bears five stamens and two styles.

Centella plants are found in tropical and subtropical regions of the world. For example, Centella asiatica commonly grows throughout India, Sri Lanka, northern Australia, Indonesia, Malaysia, and other parts of Asia.

Illustrative species include, for example, Centella asiatica, Centella erecta, and the like. Centella extracts may be prepared using methods known to those skilled in the art. Centella extracts may be present in the compositions of this disclosure at a concentration of at least about 0.001%, preferably at least about 1%, and more preferably at least about 5%. The concentration of Centella extracts in the compositions of this disclosure typically is not greater than about 20% or about 30%, and usually is not greater than about 10% or about 15%.

The extracts of the genus Centella according to this disclosure generally contain ingredients from the group consisting of triterpene acids and triterpene glycosides. Illustrative triterpene acids include, for example, asiatic acid, brahmic acid, isobrahmic acid, madecassic acid, betulinic acid, and the like. Illustrative triterpene glycosides include, for example, asiaticoside, madecassoside, brahmoside, brahminoside, and the like. These ingredients differ in composition according to the starting material and extraction method selected.

Mixtures of Centella plant extracts may also be useful in this disclosure. Mixtures of ingredients, e.g., individual molecular compositions, from one or more Centella plant extracts may further be useful in this disclosure.

Jatropha is a member of the Euphorbiaceae family. It is commonly known as Barbados nut, Purging nut or Physic nut. Jatropha plants have large green to pale-green leaves. Male and female flowers are produced on the same inflorescence, averaging about 10 male flowers to each female flower. Fruits are produced in the winter, or there may be several crops during the year if soil moisture is good and temperatures are sufficiently high. The seeds are mature when the capsule changes from green to yellow.

Jatropha plants are found in tropical and subtropical regions of the world. The plant can grow in wastelands and grows on almost any terrain, even on gravelly, sandy and saline soils. For example, Jatropha plants, e.g., Jatropha curcas, commonly grow throughout India, Sri Lanka, northern Australia, Indonesia, Malaysia, and other parts of Asia, as well as Africa, Central America and Mexico.

Illustrative species include, for example, Jatropha curcas, Jatropha gossypifolia, and the like. Jatropha extracts may be prepared using methods known to those skilled in the art. Jatropha extracts may be present in the compositions of this disclosure at a concentration of at least about 0.001%, preferably at least about 1%, and more preferably at least about 5%. The concentration of Jatropha extracts in the compositions of this disclosure typically is not greater than about 20% or about 30%, and usually is not greater than about 10% or about 15%.

The extracts of the genus Jatropha according to this disclosure generally contain ingredients from the groups consisting of oils, lectins and saponins. These ingredients differ in composition according to the starting material and extraction method selected.

Mixtures of Jatropha plant extracts may also be useful in this disclosure. Mixtures of ingredients, e.g., individual molecular compositions, from one or more Jatropha plant extracts may further be useful in this disclosure.

Aegle (Correa) is a member of the Rutaceae family. It is commonly known as Bael. Aegle includes mid-sized, slender, aromatic, gum-bearing trees growing up to 18 meters tall. The Bael fruit has a smooth, woody shell with a green, gray, or yellow peel. The plant parts useful in this disclosure include the whole fruit, tender and/or ripen, outer hard cover, leaves, roots and branches.

Aegle plants are found in dry forests on hills and plains of northern, central and southern India, southern Nepal, Vietnam and Thailand. It is cultivated throughout India.

Illustrative species include, for example, Aegle marmelos, and the like. Aegle extracts may be prepared using methods known to those skilled in the art. Aegle extracts may be present in the compositions of this disclosure at a concentration of at least about 0.001%, preferably at least about 1%, and more preferably at least about 5%. The concentration of Aegle extracts in the compositions of this disclosure typically is not greater than about 20% or about 30%, and usually is not greater than about 10% or about 15%.

The extracts of the genus Aegle according to this disclosure generally contain marmalosin from the fruit, coumarin from young bark, umbelliferone and coumarin from old bark, and d-limonene from leaves. Mature bark contains fragrine, umbelliferone marmesin, sterols and triterpenoids.

Mixtures of Aegle plant extracts may also be useful in this disclosure. Mixtures of ingredients, e.g., individual molecular compositions, from one or more Aegle plant extracts may further be useful in this disclosure.

Terminalia is a member of the Combretaceae family. Terminalia includes large trees (deciduous) of the flowering plant variety. The deciduous trees can grow up to 30 meters tall and have a trunk up to 1 meter in diameter. The leaves are alternate to subopposite in arrangement and oval in shape. The fruit is drupe-like and brackish having 5 longitudinal ridges. The leaves appear at the very tips of the shoots. The plant parts useful in this disclosure include the whole fruit, tender and/or ripen, dry or raw, outer hard cover of the tender and/or ripen fruit.

Terminalia plants are found in tropical and subtropical regions of the world. For example, Terminalia plants, e.g., Terminalia chebula and Terminalia bellerica, commonly grow throughout India, Sri Lanka, Vietnam, Malaysia, and other parts of Asia.

Illustrative species include, for example, Terminalia chebula, Terminalia bellerica, and the like. Terminalia extracts may be prepared using methods known to those skilled in the art. Terminalia extracts may be present in the compositions of this disclosure at a concentration of at least about 0.001%, preferably at least about 1%, and more preferably at least about 5%. The concentration of Terminalia extracts in the compositions of this disclosure typically is not greater than about 20% or about 30%, and usually is not greater than about 10% or about 15%.

The extracts of the genus Terminalia according to this disclosure generally contain ingredients from the groups consisting of cyclic triterpenes and their derivatives, flavonoids, tannins, and other aromatics. Other illustrative ingredients include gallic acid, ellagic acid, ethyl gallate, and the like.

Mixtures of Terminalia plant extracts may also be useful in this disclosure. Mixtures of ingredients, e.g., individual molecular compositions, from one or more Terminalia plant extracts may further be useful in this disclosure.

Phyllanthus is a member of the Phyllanthaceae family. Leafflower is the common name for all Phyllanthus species. Phyllanthus has a large variety of growth forms including annual and perennial herbs, shrubs, and the like. The plant parts useful in this disclosure include the whole fruit, tender and/or ripen, dry or raw, outer hard cover of the tender and/or ripen fruit.

Phyllanthus plants are found in tropical and subtropical regions of the world. For example, Phyllanthus plants, e.g., Phyllanthus emblica, commonly known as the Indian gooseberry (aamla) grow throughout India, Sri Lanka, northern Australia, Indonesia, Malaysia, as well as other parts of Asia.

Illustrative species include, for example, Phyllanthus emblica, and the like. Phyllanthus emblica is also known as Emblica officinalis. Phyllanthus extracts may be prepared using methods known to those skilled in the art. Phyllanthus extracts may be present in the compositions of this disclosure at a concentration of at least about 0.001%, preferably at least about 1%, and more preferably at least about 5%. The concentration of Phyllanthus extracts in the compositions of this disclosure typically is not greater than about 20% or about 30%, and usually is not greater than about 10% or about 15%.

The extracts of the genus Phyllanthus according to this disclosure generally contain ingredients from the groups consisting of lignans (e.g., phyllanthin and hypophyllanthin), tannins, and pectins. Other illustrative ingredients include ascorbic acid, and the like.

Mixtures of Phyllanthus plant extracts may also be useful in this disclosure. Mixtures of ingredients, e.g., individual molecular compositions, from one or more Phyllanthus plant extracts may further be useful in this disclosure.

Spilanthes is a member of the Asteraceae family. Spilanthes acmella (Acmella oleracea) is commonly known as the toothache plant and Paracress. The Indian name is Akarkara. It grows well in full sun to partial shade reaching a height of 12 to 15 inches with a spread of 24 to 30 inches. It has bronze-green foliage with yellow petaless flowers with a red eye on top of long stems. The plant parts useful in this disclosure include roots, leaves and flowers.

Spilanthes plants are found in tropical and subtropical regions of the world. Spilanthes plants are native to the tropics of Africa and South America. The plants are also found in Southeast Asia and India.

Illustrative species include, for example, Spilanthes acmella (Acmella oleracea), and the like. Spilanthes acmella is also known as Acmella oleracea. Spilanthes extracts may be prepared using methods known to those skilled in the art. Spilanthes extracts may be present in the compositions of this disclosure at a concentration of at least about 0.001%, preferably at least about 1%, and more preferably at least about 5%. The concentration of Spilanthes extracts in the compositions of this disclosure typically is not greater than about 20% or about 30%, and usually is not greater than about 10% or about 15%.

The extracts of the genus Spilanthes according to this disclosure generally contain ingredients from the groups consisting of alkylamides and triterpenes. Other illustrative ingredients include spilanthol, stigmasterol, and the like.

Mixtures of Spilanthes plant extracts may also be useful in this disclosure. Mixtures of ingredients, e.g., individual molecular compositions, from one or more Spilanthes plant extracts may further be useful in this disclosure.

Mixtures of one or more Bacopa plant extracts, Centella plant extracts, Jatropha plant extracts, Aegle plant extracts, Terminalia plant extracts, Phyllanthus plant extracts, and/or Spilanthes plant extracts, in any permissible combination may also be useful in this disclosure. For example, mixtures of at least one Bacopa plant extract, at least one Centella plant extract, and at least one Jatropha plant extract, or mixtures of at least one Bacopa plant extract and at least one Centella plant extract, or mixtures of at least one Bacopa plant extract and at least one Jatropha plant extract, or mixtures of at least one Centella plant extract and at least one Jatropha plant extract, may also be useful in this disclosure. Mixtures of ingredients, e.g., individual molecular compositions, from one or more Bacopa plant extracts, Centella plant extracts, Jatropha plant extracts, Aegle plant extracts, Terminalia plant extracts, Phyllanthus plant extracts, and/or Spilanthes plant extracts, in any permissible combination may further be useful in this disclosure. For example, mixtures of ingredients of at least one Bacopa plant extract, at least one Centella plant extract, and at least one Jatropha plant extract, or mixtures of at least one Bacopa plant extract and at least one Centella plant extract, or mixtures of at least one Bacopa plant extract and at least one Jatropha plant extract, or mixtures of at least one Centella plant extract and at least one Jatropha plant extract, may also be useful in this disclosure.

The extracts to be used in accordance with this disclosure may be prepared by known methods of extracting plants or parts thereof. Particulars of suitable conventional extraction processes, such as maceration, remaceration, digestion, agitation maceration, vortex extraction, ultrasonic extraction, countercurrent extraction, percolation, repercolation, evacolation (extraction under reduced pressure), diacolation and solid/liquid extraction under continuous reflux in a Soxhlet extractor, which are familiar to those skilled in the art and which may all be used. Fresh plants or parts thereof are suitable as the starting material although dried plants and/or plant parts that may be mechanically size-reduced before extraction are normally used. Any size reduction methods known to those skilled in the art, for example crushing in a mortar, may be used. Raw plants and plant products may also be used.

Preferred solvents for the extraction process are organic solvents, water (distilled or non-distilled, with a temperature from about 4° C. to about 80° C.) or mixtures of organic solvents and water, more particularly low molecular weight alcohols, esters, hydrocarbons, ketones or halogenated hydrocarbons with more or less large water contents. Extraction with water, methanol, ethanol, pentane, hexane, heptane, acetone, propylene glycols, polyethylene glycols, ethyl acetate, dichloromethane, trichloromethane and mixtures thereof is particularly preferred. The extraction process is generally carried out at 20 to 100° C., preferably at 25 to 90° C., and more particularly at 30 to 80° C. Liquid CO₂ extraction may also be used.

In one embodiment, the extraction process is carried out in an inert gas atmosphere to avoid oxidation of the ingredients of the extract. The extraction times are selected by the expert in dependence upon the starting material, the extraction process, the extraction temperature and the ratio of solvent to raw material, etc. After the extraction process, the crude extracts obtained may optionally be subjected to other typical steps, such as for example purification, concentration and/or decoloration. If desired, the extracts thus prepared may be subjected, for example, to the selective removal of individual unwanted ingredients. The extraction process may be carried out to any degree, but is usually continued to exhaustion. Typical yields (equal to extract dry matter, based on the quantity of raw material used) in the extraction of dried plants are in the range from 3 to 20 and more particularly 4 to 16% by weight. The extraction conditions and the yields of the final extracts may be selected according to the desired application. If desired, the extracts may then be subjected, for example, to spray drying or freeze drying.

Other extraction processes useful in this disclosure include, for example, super critical carbon dioxide extraction, pure carbon dioxide extraction, and oil extraction. Such extraction processes can be carried out by conventional procedures known in the art.

This disclosure also relates to cosmetic and/or pharmaceutical preparations that contain an extract selected from the group consisting of a Bacopa plant extract, a Centella plant extract, a Jatropha plant extract, an Aegle plant extract, a Terminalia plant extract, a Phyllanthus plant extract, a Spilanthes plant extract, and mixtures thereof.

Illustrative cosmeceutical compositions include, for example, sunless tanning compositions, sunscreen compositions, ultraviolet radiation protective compositions, anti-aging compositions, anti-wrinkle compositions, moisturizer compositions, skin soothing compositions, skin/hair softening compositions, skin/hair treating compositions, anti-inflammatory compositions, cosmeceutical preservative compositions, cosmeceutical stabilizer compositions, antioxidant compositions, free radical inhibitive compositions, cleansing compositions, anti-microbial compositions, anti-fungal compositions, hair coloring compositions, and oral cleaning/deodoring compositions.

Cosmetic and/or pharmaceutical preparations based on the Bacopa plant, the Centella plant, the Jatropha plant, the Aegle plant, the Terminalia plant, the Phyllanthus plant, and/or the a Spilanthes plant, and/or mixtures thereof, show surprisingly good skin and/or hair care and protecting properties against stress and against environmental influences coupled with high dermatological compatibility. In addition, the preparations are effective moisture-regulating skin moisturizers. The preparations are also distinguished by a high antioxidation capacity that, on the one hand, protects the skin against inflammatory reactions and against oxidation-induced skin aging processes; on the other hand, cosmetic preparations are simultaneously protected against oxidative degradation (deterioration). In addition, the products thus obtained are capable of preventing damage to human fibroblasts and keratinocytes by UV radiation and may therefore be used as sun protection factors in cosmetics. The preparations are useful in antioxidative stress applications and also the repair of damaged skin and hair (both UV and normal damaged).

The quantity of plant extracts used in the preparations mentioned is governed by the concentration of the individual ingredients and by the way in which the extracts are used. In general, the plant extract is used in a quantity based on the final cosmetic and/or pharmaceutical preparation, for example, an amount of from about 0.001 to about 25% by weight, preferably about 0.01 to about 10% by weight, and more particularly about 0.1 to about 5% by weight, with the proviso that the quantities add up to 100% by weight with other auxiliaries and additives and with water. The total content of auxiliaries and additives may be from about 1 to about 50% by weight and is preferably about 5 to about 40% by weight, based on the final cosmetic and/or pharmaceutical preparation. The preparations may be produced by conventional processes known in the art.

This disclosure also relates to the manifold use of Bacopa plant extracts (e.g., Bacopa monniera plant extracts), Centella plant extracts (e.g., Centella asiatica plant extracts), Jatropha plant extracts (e.g., Jatropha curcas), Aegle plant extracts (e.g., Aegle marmelos), Terminalia plant extracts (e.g., Terminalia chebula and Terminalia bellerica), Phyllanthus plant extracts (e.g., Phyllanthus emblica or Emblica officinalia), and/or Spilanthes plant extracts (e.g., Spilanthes acmella), and/or mixtures thereof, for example in skin and/or hair care preparations, particularly against stress; in moisture-regulating moisturizers. The preparations are particularly useful in antioxidative stress applications and also the repair of damaged skin and hair (both UV and normal damaged).

Care preparations in the context of this disclosure are understood to be skin and/or hair care preparations. These care preparations have a cleaning and restoring effect and show moisture-regulating and ultraviolet (UV) protection properties. In principle, the extracts according to this disclosure may be used in a variety of cosmeceutical products.

This disclosure provides a sunless tanning composition having a tanning active system in an amount effective for sunless tanning. The sunless tanning composition may optionally have a moisturizing system in an amount effective for moisturizing skin.

The sunless tanning composition according to this disclosure can be essentially oil-free. In one embodiment, the sunless tanning composition is oil-free. The term “oil-free”, as used herein, is defined as free of mineral oil and/or petrolatum. By formulating the sunless tanning compositions as oil-free, the compositions are non-irritating to sensitive skin, have an enhanced skin-feel when applied, and/or are less occlusive to the tanning actives. The sunless tanning compositions of this disclosure can also be prepared with oil.

The sunless tanning active system of this disclosure includes one or more sunless tanning actives for imparting a tan to the skin. Suitable tanning actives for use in the sunless tanning active system of this disclosure include at least one Bacopa plant extract, Centella plant extract, Jatropha plant extract, Aegle plant extract, Terminalia plant extract, Phyllanthus plant extract, Spilanthes plant extract, and/or mixtures thereof. Other tanning actives that may be present in combination with the Bacopa plant extract, Centella plant extract, Jatropha plant extract, Aegle plant extract, Terminalia plant extract, Phyllanthus plant extract, Spilanthes plant extract, and/or mixtures thereof include, for example, dihydroxyacetone (DHA), erythrulose, melanin, mahakanni, or any combinations thereof. In an embodiment, the tanning actives are at least one Bacopa plant extract, Centella plant extract, Jatropha plant extract, Aegle plant extract, Terminalia plant extract, Phyllanthus plant extract, Spilanthes plant extract, and/or mixtures thereof and DHA.

The mixtures can include, for example, mixtures of Bacopa plant extracts, or mixtures of ingredients, e.g., individual molecular compositions, from one or more Bacopa plant extracts; mixtures of Centella plant extracts, or mixtures of ingredients, e.g., individual molecular compositions, from one or more Centella plant extracts; mixtures of Jatropha plant extracts, or mixtures of ingredients, e.g., individual molecular compositions, from one or more Jatropha plant extracts; mixtures of Aegle plant extracts, or mixtures of ingredients, e.g., individual molecular compositions, from one or more Aegle plant extracts; mixtures of Terminalia plant extracts, or mixtures of ingredients, e.g., individual molecular compositions, from one or more Terminalia plant extracts; mixtures of Phyllanthus plant extracts, or mixtures of ingredients, e.g., individual molecular compositions, from one or more Phyllanthus plant extracts; and mixtures of Spilanthes plant extracts, or mixtures of ingredients, e.g., individual molecular compositions, from one or more Spilanthes plant extracts. Mixtures of one or more Bacopa plant extracts, Centella plant extracts, Jatropha plant extracts, Aegle plant extracts, Terminalia plant extracts, Phyllanthus plant extracts, and/or Spilanthes plant extracts, in any permissible combination, or mixtures of ingredients, e.g., individual molecular compositions, from one or more Bacopa plant extracts, Centella plant extracts, Jatropha plant extracts, Aegle plant extracts, Terminalia plant extracts, Phyllanthus plant extracts, and/or Spilanthes plant extracts, in any permissible combination, may also be useful in this disclosure.

For example, mixtures of at least one Bacopa plant extract, at least one Centella plant extract, and at least one Jatropha plant extract, or mixtures of at least one Bacopa plant extract and at least one Centella plant extract, or mixtures of at least one Bacopa plant extract and at least one Jatropha plant extract, or mixtures of at least one Centella plant extract and at least one Jatropha plant extract, may also be useful in this disclosure. Mixtures of ingredients of at least one Bacopa plant extract, at least one Centella plant extract, and at least one Jatropha plant extract, or mixtures of at least one Bacopa plant extract and at least one Centella plant extract, or mixtures of at least one Bacopa plant extract and at least one Jatropha plant extract, or mixtures of at least one Centella plant extract and at least one Jatropha plant extract, may also be useful in this disclosure.

The sunless tanning active system is present in the sunless tanning compositions of this disclosure in an amount effective for imparting the desired tanning effect in the desired time. A suitable effective amount of the tanning active system is about 0.001 weight % to about 25 weight %, based on the total weight of the sunless tanning composition. In an embodiment, the tanning active system is present in an amount about 0.01 weight % to about 15 weight %, based on the total weight of the composition. In yet another embodiment, the tanning active system is present in an amount about 0.1 weight % to about 10 weight %, based on the total weight of the composition.

The moisturizing system of the sunless tanning compositions can be formulated to provide substantial moisturizing to the skin, including simultaneously with the tanning of the skin and even after the tanning has been completed. It has been found that substantial moisturizing to the skin can be achieved by the moisturizing system of this disclosure without the inclusion of mineral oil and/or petrolatum. As a result, a composition that is less irritating to sensitive skin is formulated. Additionally, the absence of the mineral oil and petrolatum results in a less occlusive composition.

The moisturizing system can include at least one Bacopa plant extract, Centella plant extract, Jatropha plant extract, Aegle plant extract, Terminalia plant extract, Phyllanthus plant extract, Spilanthes plant extract, and/or mixtures thereof, and optionally one or more moisturizing components suitable for providing the desired moisturizing effects in the absence of mineral oil and petrolatum. In an embodiment, the moisturizing system includes two or more moisturizing components. In yet another embodiment, the moisturizing system includes at least three moisturizing components.

Suitable optional moisturizing components include, but are not limited to, one or more polyols, siloxanes, naturally occurring fats and oils, or any combinations thereof.

The one or more polyols that may be used as moisturizing components include, but are not limited to, glycerin, propylene glycol, butylene glycol, hexylene glycol, pentylene glycol, caprylyl glycol, sorbitol, or any combinations thereof.

The one or more siloxanes that may be used as moisturizing components in this disclosure include, but are not limited to, dimethicone, cyclomethicone, phenyl trimethicone, phenyl dimethicone, cetyl dimethicone, stearyl dimethicone, amodimethicone, C₃₀₋₄₅ alkyl dimethicone, C₃₀₋₄₅ alkyl methicone, cetearyl methicone, dimethicone copolyol, cyclopentasiloxane, or any combinations thereof.

The one or more naturally occurring fats and oils that may be used as moisturizing components in this disclosure include, but are not limited to, shea butter, shea butter oil, cocoa butter, jojoba butter, aloe butter, olive butter, coconut oil, jojoba oil, olive oil, sunflower seed oil, meadowfoam seed oil, macadamia nut oil, sesame oil, borage seed oil, or any combinations thereof.

The moisturizing system can be present in the sunless tanning composition in an amount about 0.001 weight % to about 25 weight %, based on the total weight of the composition. In an embodiment, the moisturizing system is present in an amount about 0.01 weight % to about 15 weight %, based on the total weight of the composition. In yet another embodiment, the moisturizing system is present in an amount about 0.1 weight % to about 10 weight %, based on the total weight of the composition.

In one embodiment of the present disclosure, by including one or more polyols, one or more siloxanes, and one or more naturally occurring fats and oils with the at least one Bacopa plant extract, Centella plant extract, Jatropha plant extract, Aegle plant extract, Terminalia plant extract, Phyllanthus plant extract, Spilanthes plant extract, and/or mixtures thereof, in the moisturizing system, the resulting sunless tanning composition with this moisturizing system can have the desired moisturizing properties, while being non-irritating and/or non-occlusive. Also, the composition may unexpectedly have an enhanced skin-feel.

In an embodiment, the one or more polyols, one or more siloxanes, and one or more naturally occurring fats and oils should be present in a weight ratio of about 20:5:1 to about 7:2:1. In yet another embodiment, the moisturizing system has dimethicone, glycerin and shea butter present in a ratio of about 14:4.4:1 to about 12:2.5:1. This particular embodiment, when used to formulate a sunless tanning composition according to this disclosure, provides enhanced moisturizing properties to the composition.

The sunless tanning compositions according to this disclosure may further include one or more amphoglycinates (amphoacetates). Suitable amphoglycinates include, but are not limited to sodium olive amphoacetate, sodium sunflowerseed amphoacetate, sodium cocoabutter amphoacetate, sodium sesame amphoacetate, sodium sweetalmond amphoacetate, sodium caproamphoacetate, sodium capryloamphoacetate, sodium isostearoamphoacetate, sodium lauroamphoacetate, sodium myristoamphoacetate, sodium oleoamphoacetate, sodium palmamphoacetate, sodium peanut amphoacetate, sodium ricinoleoamphoacetate, sodium stearoamphoacetate, sodium tallow amphoacetate, sodium undecylenamphoacetate, sodium wheat germamphoacetate, sodium cocoyl glycinate, or any mixtures thereof. These compounds are generally available from a variety of sources or can be prepared by methods known in the art.

The amphoglycinate may be present in the sunless tanning composition in an amount about 1 weight % to about 30 weight %, based on the total weight of the composition.

The sunless tanning composition may further include one or more additional components, including, but not limited to, sugars, keto-sugars, surface-active agents, polymers, softening agents, moisturizers, water-proofing agents, vitamins, sweet orange citroflavonoids, hesperitine, ozone stressed yeast lysate, Saccharomyces cerevisiae ferment filtrate lysate, methylsulfonyl methane (MSM), also known as dimethyl sulfone and methyl sulfone, UV filters, skin penetration agents, or any combinations thereof. These one or more additional components may be present in an amount up to about 30 weight % of the total weight of the composition.

Suitable UV filters may include, but are not limited to, dibenzoylmethane, oxybenzone, sulisobenzone, dioxybenzone, menthyl anthranilate, para aminobenzoic acid (PABA) ester, benzophenone-3, butyldibenzoylmethane, dimethyl cinnamate, octyl methoxycinnamate, DEA methoxycinnamate, octocrylene, drometrizole trisiloxane, octyl salicylate, homomethyl salicylate, octyl dimethyl PABA, TEA salicylate, 4-methyl benzylidene camphor, 3-benzylidene camphor, benzylidene camphor sulfonic acid ester, octyl triazone, phenyl benzimidazole sulfonic acid ester, terephthalydiene dicamphor sulfonic acid ester, di-t-butyl hydroxybenzylidene camphor, ethyl PABA, butylmethoxy dibenzoylmethane (avobenzone), terephthalydiene methylene bis-benzotriazoyltetramethylbutyl-phenol, diethylhexyl-2,6-naphthalate, bis-ethylhexyloxyphenol methoxyphenol triazine, hydroxy methylphenyl benzotriazole, methylene bis-benzotriazoyltetramethylbutylphenol, bis-ethylhexyloxyphenol methoxyphenol triazine, hydroxybenzophenone, a benzotriazole, a dibenzoyl methane, an oxanilide, a hydroxy cinnamate, oil dispersible titanium dioxide, oil dispersible zinc oxide, a silicone-anchored sunscreen, para aminobenzoic acid (PABA), salicylic acid, TEA salicylate, benzylidene camphor sulfonic acid, phenyl benzimidazole sulfonic acid, terephthalydiene dicamphor sulfonic acid, hydroxy cinnamic acid, any derivatives thereof, or any combinations thereof.

Other suitable additives may include, but are not limited to, antioxidants, such as, erythrobic acid, sodium metabisulfite, sodium sulfite, rosemary extract, tocopherol, a derivative of tocopherol including a tocotriene, carotene, a carotenoid, lutein or lutein ester, a carotenoid, a phenolic antioxidant, a bioflavonoid, a plant extract, or any combinations thereof; keratolytic agents, such as, salicylic acid, resorcinol, peroxide of an organic acid, or any combinations thereof; anti-inflammatory agents, such as, steroidal and non-steroidal anti-inflammatory agents, plant extracts that have demonstrated anti-inflammatory activity, or any combinations thereof; vitamins, such as, Vitamin K, Vitamin C, retinol (vitamin A), tocopherol, or any combinations thereof; emollients, such as, cetearyl octanoate, octyl palmitate, glyceryl monostearate, caprylic triglyceride, capric triglyceride, or any combinations thereof; humectants, such as, hyaluronic acid, one or more derivatives of hyaluronic acid, or any combinations thereof; skin penetration enhancers, such as, ozone, SEPA, butylene glycol, cis-isomer of an unsaturated fatty acid, or any combinations thereof; emulsifiers, such as, glyceryl stearate, cetearyl alcohol, cetyl alcohol, PEG-40 stearate, or any combinations thereof; thickening agents, such as, xanthan gum, carbomer, clay, hydroxyethyl cellulose, or any combinations thereof; preservatives, such as, an alkyl paraben, an alcohol, salts of benzoic acid, salts of sorbic acid, or any combinations thereof; colorants, such as, synthetic and natural colorants, henna, caramel, photo-chromic and thermo-chromic colorants and pigments, surface-treated or hydrophobically modified colorants, or any combinations thereof; organic acids and their derivatives, such as citric acid, glycolic acid, glutamic acid, gluco delta lactone, or any combinations thereof; chelating agents, such as, disodium EDTA; pH adjusters, such as, an acid, a base, a buffer, or any combinations thereof, to adjust and maintain the pH to about 3.0 to about 7.5; fragrances; proteins; peptides; and amino acids.

The sunless tanning composition is applicable to a variety of personal care product forms including, but not limited to body wash, bar soap, liquid soap, lather, skin and/or hair care preparation, cream, foam, gel, lotion, solution, emulsion, pomade, mousse, balm, stick, pump spray, aerosol spray, or any combinations thereof.

The sunless tanning composition can be organic solvent based, water based or it can be an emulsion, including, but not limited to, an oil-in-water emulsion or a water-in-oil emulsion.

This disclosure provides a method of simultaneously sunless tanning and moisturizing skin having the step of topically applying to the skin an effective amount of a sunless tanning composition having the tanning active system and moisturizing system.

The preparations according to this disclosure have an excellent skin and/or hair care effect coupled with high dermatological compatibility. They also show high stability, particularly against oxidative decomposition of the products.

This disclosure also relates to the use of the extracts of the Bacopa plant, Centella plant, Jatropha plant, Aegle plant, Terminalia plant, Phyllanthus plant, Spilanthes plant, and/or mixtures thereof, in sun protection preparations, e.g., sunscreens. Sun protection factors or UV protection factors in the context of this disclosure are light protection factors that are useful in protecting human skin and/or hair against harmful effects of direct and indirect solar radiation. The ultraviolet radiation of the sun responsible for tanning of the skin is divided into the sections UV-C (wavelengths 200-280 nm), UV-B (280-315 nm) and UV-A (315-400 nm).

The pigmenting of normal skin under the influence of solar radiation, i.e. the formation of melanins, is differently effected by UV-B and UV-A. Exposure to UV-A (long-wave UV) results in darkening of the melanins already present in the epidermis without any sign of harmful effects. It is different with so-called short-wave UV (UV-B). This promotes the formation of so-called late pigment through the reformation of melanins. However, before the (protective) pigment is formed, the skin is exposed to the unfiltered radiation which, depending on the exposure time, can lead to reddening of the skin (erythema), inflammation of the skin (sunburn) or even blisters.

Extracts of the Bacopa plant, Centella plant, Jatropha plant, Aegle plant, Terminalia plant, Phyllanthus plant, Spilanthes plant, and/or mixtures thereof, are used as UV absorbers or light filters that convert UV radiation into harmless heat. They may additionally be present in combination with other sun protection factors or UV protection factors.

These other UV protection factors are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances: 3-benzylidene camphor or 3-benzylidene norcamphor and derivatives thereof, for example 3-(4-methylbenzylidene)-camphor as described in EP-B1 0693471; 4-aminobenzoic acid derivatives, preferably 4-(dimethylamino)-benzo-ic acid-2-ethylhexyl ester, 4-(dimethylamino)-benzoic acid-2-octyl ester and 4-(dimethylamino)-benzoic acid amyl ester; esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (octocrylene); esters of salicylic acid, preferably salicylic acid-2-ethylhexyl ester, salicylic acid-4-isopropylbenzyl ester, salicylic acid homomethyl ester; derivatives of benzophenone, preferably 2-hydroxy-4-methoxybenzophe-none, 2-hydroxy-4-methoxy-4′-methylbenzophenone, 2,2′-dihydroxy-4-methoxyb-enzophenone; esters of benzalmalonic acid, preferably 4-methoxybenzmalonic acid di-2-ethylhexyl ester; triazine derivatives such as, for example, 2,4,6-trianilino-(p-carb-o-2′-ethyl-1′-hexyloxy)-1,3,5-triazine and octyl triazone or dioctyl butamido triazone; propane-1,3-diones such as, for example, 1-(4-tert.butylphenyl)-3-(-4′-methoxyphenyl)-propane-1,3-dione; ketotricyclo(5.2.1.0)decane derivatives.

Suitable water-soluble substances are 2-phenylbenzimidazole-5-sulfonic acid and alkali metal, alkaline earth metal, ammonium, alkylammonium, alkanolammonium and glucammonium salts thereof; sulfonic acid derivatives of benzophenones, preferably 2-hydroxy-4-methoxybenzophenone-5-sulfonic acid and salts thereof; sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl-)-propane-1,3-dione, 4-tert.butyl-4′-methoxydibenzoyl methane or 1-phenyl-3-(4′-isopropylphenyl)-propane-1,3-dione and the enamine compounds. The UV-A and UV-B filters may of course also be used in the form of mixtures. Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminum and cerium and mixtures thereof. Silicates (talcum), barium sulfate and zinc stearate may be used as salts. The oxides and salts are used in the form of the pigments for skin and/or hair care and skin and/or hair protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Suitable hydrophobic coating materials are, above all, silicones and, among these, especially trialkoxyoctylsilanes or dimethicones. So-called micro- or nanopigments are preferably used in sun protection products. Micronized zinc oxide is preferably used.

For sunscreen preparations, the Bacopa plant extracts, Centella plant extracts, Jatropha plant extracts, Aegle plant extracts, Terminalia plant extracts, Phyllanthus plant extracts, Spilanthes plant extracts, and/or mixtures thereof, may be present in an amount effective to impart a sunscreen booster effect in the sunscreen composition. The Bacopa plant extracts, Centella plant extracts, Jatropha plant extracts, Aegle plant extracts, Terminalia plant extracts, Spilanthes plant extracts, plant extracts, and/or mixtures thereof, can increase the effectiveness of conventional sunscreen preparations in protecting human skin and/or hair against harmful effects of direct and indirect solar radiation.

The sunscreen actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the sunscreen compositions of this disclosure in an amount effective for imparting the desired sunscreen effect in the desired time. A suitable effective amount of the sunscreen actives is about 0.001 weight % to about 25 weight %, based on the total weight of the sunscreen composition. In an embodiment, the sunscreen actives are present in an amount of from about 0.01 weight % to about 15 weight %, based on the total weight of the composition. In yet another embodiment, the sunscreen actives are present in an amount of from about 0.1 weight % to about 10 weight %, based on the total weight of the composition.

This disclosure also relates to the use of extracts of the Bacopa plant, Centella plant, Jatropha plant, Aegle plant, Terminalia plant, Phyllanthus plant, Spilanthes plant, and/or mixtures thereof, in preparations against fibroblast and/or keratinocyte damage by UV-A radiation and/or UV-B radiation and as anti-inflammatory additives.

UV-A rays penetrate into the dermis where they lead to oxidative stress that is demonstrated by lipoperoxidation of the cytoplasm membranes. The lipoperoxides are degraded to malonaldialdehyde (MDA) which will crosslink many biological molecules, such as proteins and nuclein bases (enzyme inhibition or mutagenesis). The extracts of the Bacopa plant, Centella plant, Jatropha plant, Aegle plant, Terminalia plant, Phyllanthus plant, Spilanthes plant, and/or mixtures thereof, according to this disclosure significantly reduce the level of MDA in human fibroblasts induced by UV-A rays and thus show a high capacity for reducing the harmful effects of oxidative stress on the skin.

UV-B rays initiate inflammation by activating an enzyme, namely phospholipase A2 or PLA2. This inflammation (erythema, odema) is induced by the removal of arachidonic acid from the phospholipids of the plasma membrane by the phospholipase. Arachidonic acid is the precursor of the prostaglandins which cause inflammation and cell membrane damage. The prostaglandins E2 (PGE2) are formed by cyclooxygenase. The degree of release of the cytoplasm enzyme LDH (lactate dehydrogenase) in human keratinocytes serves as a marker for cell damage.

The extracts of the Bacopa plant, Centella plant, Jatropha plant, Aegle plant, Terminalia plant, Phyllanthus plant, Spilanthes plant, and/or mixtures thereof, reduce the effect of UV-B radiation on the number of keratinocytes and on the content of released LDH. Accordingly, the extracts have the ability to reduce cell membrane damage caused by UV-B radiation.

The ultraviolet radiation protective, anti-aging, anti-wrinkle actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the ultraviolet radiation protective, anti-aging, anti-wrinkle compositions of this disclosure in an amount effective for imparting the desired ultraviolet radiation protective, anti-aging, anti-wrinkle effect in the desired time. A suitable effective amount of the ultraviolet radiation protective, anti-aging, anti-wrinkle actives is about 0.001 weight % to about 25 weight %, based on the total weight of the ultraviolet radiation protective, anti-aging, anti-wrinkle composition. In an embodiment, the ultraviolet radiation protective, anti-aging, anti-wrinkle actives are present in an amount of from about 0.01 weight % to about 15 weight %, based on the total weight of the composition. In yet another embodiment, the ultraviolet radiation protective, anti-aging, anti-wrinkle actives are present in an amount of from about 0.1 weight % to about 10 weight %, based on the total weight of the composition.

Moisture-regulating moisturizers according to this disclosure are understood to be skin care preparations that are intended to regulate skin moisture. In the context of this disclosure, this conforms to the definition of a moisturizer. They are substances or mixtures of substances which provide cosmetic and/or pharmaceutical preparations with the ability to reduce the release of moisture from the stratum corneum (horny layer) after application to and spreading over the surface of the skin.

The moisturizers according to this disclosure contain extracts of the Bacopa plant, Centella plant, Jatropha plant extract, Aegle plant, Terminalia plant, Phyllanthus plant, Spilanthes plant, and/or mixtures thereof. Other moisturizers, for example, may be present in combination with the plant extract, including polyglycerol fatty acid esters based on C₁₂₋₁₈ fatty acids, for example tetraglyceryl monooleate, triglyceryl diisostearate; pyroglutamic acid or L-arginine pyroglutamate, L-lysine pyroglutamate; mixtures of amino acids such as, for example, L-alanine, L-arginine, L-serine, L-threonine; propylene glycol; acetamide; polysaccharides or hyaluronic acid; castor oil ethers and sorbitan esters.

The moisturizing actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the moisturizing compositions of this disclosure in an amount effective for imparting the desired moisturizing effect in the desired time. A suitable effective amount of the moisturizing actives is about 0.001 weight % to about 25 weight %, based on the total weight of the moisturizing composition. In an embodiment, the moisturizing actives are present in an amount of from about 0.01 weight % to about 15 weight %, based on the total weight of the composition. In yet another embodiment, the moisturizing actives are present in an amount of from about 0.1 weight % to about 10 weight %, based on the total weight of the composition.

The extracts according to this disclosure may be used as skin soothing additives for cosmetic and/or pharmaceutical preparations used in skin care. The skin soothing actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the skin soothing compositions of this disclosure in an amount effective for imparting the desired skin soothing effect in the desired time. A suitable effective amount of the skin soothing actives is about 0.001 weight % to about 25 weight %, based on the total weight of the skin soothing composition. In an embodiment, the skin soothing actives are present in an amount of from about 0.01 weight % to about 15 weight %, based on the total weight of the composition. In yet another embodiment, the skin soothing actives are present in an amount of from about 0.1 weight % to about 10 weight %, based on the total weight of the composition.

The extracts according to this disclosure may be used as skin/hair softener additives for cosmetic and/or pharmaceutical preparations used in skin/hair care. The skin/hair softener actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the skin/hair softener compositions of this disclosure in an amount effective for imparting the desired skin/hair softener effect in the desired time. A suitable effective amount of the skin/hair softener actives is about 0.001 weight % to about 25 weight %, based on the total weight of the skin/hair softener composition. In an embodiment, the skin/hair softener actives are present in an amount of from about 0.01 weight % to about 15 weight %, based on the total weight of the composition. In yet another embodiment, the skin/hair softener actives are present in an amount of from about 0.1 weight % to about 10 weight %, based on the total weight of the composition.

The extracts according to this disclosure may be used as skin/hair treatment additives for cosmetic and/or pharmaceutical preparations used in skin/hair care, e.g., treatment of acne, dermatitis, eczema, psoriasis, and other skin problems; skin whitening, and the like. The skin/hair treatment actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the skin/hair treatment compositions of this disclosure in an amount effective for imparting the desired skin/hair treating effect in the desired time. A suitable effective amount of the skin/hair treatment actives is about 0.001 weight % to about 25 weight %, based on the total weight of the skin/hair treatment composition. In an embodiment, the skin/hair treatment actives are present in an amount of from about 0.01 weight % to about 15 weight %, based on the total weight of the composition. In yet another embodiment, the skin/hair treatment actives are present in an amount of from about 0.1 weight % to about 10 weight %, based on the total weight of the composition.

In principle, the extracts according to this disclosure may be used as anti-inflammatory additives for any cosmetic and/or pharmaceutical preparations used against inflammation of the skin and hence in skin care. The inflammation of the skin may be caused by various factors.

The anti-inflammatory actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the anti-inflammatory compositions of this disclosure in an amount effective for imparting the desired anti-inflammatory effect in the desired time. A suitable effective amount of the anti-inflammatory actives is about 0.001 weight % to about 25 weight %, based on the total weight of the anti-inflammatory composition. In an embodiment, the anti-inflammatory actives are present in an amount of from about 0.01 weight % to about 15 weight %, based on the total weight of the composition. In yet another embodiment, the anti-inflammatory actives are present in an amount of from about 0.1 weight % to about 10 weight %, based on the total weight of the composition.

The extracts according to this disclosure may be used as preservatives for cosmetic and/or pharmaceutical preparations used in skin and/or hair care. The preservative actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the cosmetic and/or pharmaceutical compositions of this disclosure in an amount effective for imparting the desired preservative effect in the desired time. A suitable effective amount of the preservative actives is about 0.001 weight % to about 25 weight %, based on the total weight of the cosmetic and/or pharmaceutical composition. In an embodiment, the preservative actives are present in an amount of from about 0.01 weight % to about 15 weight %, based on the total weight of the composition. In yet another embodiment, the preservative actives are present in an amount of from about 0.1 weight % to about 10 weight %, based on the total weight of the composition.

The extracts according to this disclosure may be used as stabilizers for cosmetic and/or pharmaceutical preparations used in skin and/or hair care. The stabilizer actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the cosmetic and/or pharmaceutical compositions of this disclosure in an amount effective for imparting the desired stabilizer effect in the desired time. A suitable effective amount of the stabilizer actives is about 0.001 weight % to about 25 weight %, based on the total weight of the cosmetic and/or pharmaceutical composition. In an embodiment, the stabilizer actives are present in an amount of from about 0.01 weight % to about 15 weight %, based on the total weight of the composition. In yet another embodiment, the stabilizer actives are present in an amount of from about 0.1 weight % to about 10 weight %, based on the total weight of the composition.

In an embodiment, cosmeceutical compositions are provided to be topically applied to the skin and/or hair, and absorbed through the skin and/or hair. The cosmeceutical compositions of this disclosure can be formulated for topical administration and applied to the skin, and absorbed through the skin, so as to reduce oxidative stress, e.g., compositions having antioxidant properties that have the ability to terminate free radical chain reactions in biological systems. Oxidative stress is a result of an imbalance between antioxidative defense systems and the formation of reactive oxygen species including free radicals. Oxidative stress can damage DNA, proteins, lipids and carbohydrates and may also alter intracellular signaling processes. The damage can contribute to cell injury and death, accelerate the aging process, and promote many diseases, such as cancer, cardiovascular diseases, and Parkinson's disease. The preparations are particularly useful in antioxidative stress applications and also the repair of damaged skin and hair (both UV and normal damaged).

This disclosure also relates to the use of extracts of the Bacopa plant, Centella plant, Jatropha plant, Aegle plant, Terminalia plant, Phyllanthus plant, Spilanthes plant, and/or mixtures thereof, as antioxidants or radical traps. Antioxidants in the context of this disclosure are oxidation inhibitors that can be isolated from the Bacopa plant, Centella plant, Jatropha plant, Aegle plant, Terminalia plant, Phyllanthus plant, Spilanthes plant, and/or mixtures thereof. Antioxidants are capable of inhibiting or preventing changes caused by the effects of oxygen and other oxidative processes in the substances to be protected. The effect of antioxidants consists mainly in their acting as radical traps for the free radicals occurring during autoxidation. The extracts of the Bacopa plant, Centella plant, Jatropha plant, Aegle plant, Terminalia plant, Phyllanthus plant, Spilanthes plant, and/or mixtures thereof, exhibit desirable free radical scavenging activity.

Besides the use of extracts of the Bacopa plant, Centella plant, Jatropha plant, Aegle plant, Terminalia plant, Phyllanthus plant, Spilanthes plant, and/or mixtures thereof, as antioxidants, other already known antioxidants may also be used. One possible use of the antioxidants, for example in cosmetic and/or pharmaceutical preparations, is their use as secondary sun protection factors because antioxidants are capable of interrupting the photochemical reaction chain which is initiated when UV rays penetrate into the skin. Besides the plant extract according to this disclosure, typical examples are amino acids (for example glycine, alanine, arginine, serine, threonine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene, lutein) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, boldin, boldo extract, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxid-dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of this disclosure (salts, esters, ethers, sugars, nucleotides, nucleosides, peptides and lipids).

The UV protection factors or antioxidants, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the UV protection/antioxidant compositions of this disclosure in an amount effective for imparting the desired UV protection/antioxidant effect in the desired time. A suitable effective amount of the UV protection/antioxidant actives is about 0.001 weight % to about 25 weight %, based on the total weight of the UV protection/antioxidant composition. In an embodiment, the UV protection/antioxidant actives are present in an amount of from about 0.01 weight % to about 15 weight %, based on the total weight of the composition. In yet another embodiment, the UV protection/antioxidant actives are present in an amount of from about 0.1 weight % to about 10 weight %, based on the total weight of the composition.

The extracts according to this disclosure may be used as cleansing additives for cosmetic and/or pharmaceutical preparations used in skin and/or hair care, e.g., hair, skin, body and hand cleansers. The cleansing actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the cleansing compositions of this disclosure in an amount effective for imparting the desired cleansing effect in the desired time. A suitable effective amount of the cleansing actives is about 0.001 weight % to about 25 weight %, based on the total weight of the cleansing composition. In an embodiment, the cleansing actives are present in an amount of from about 0.01 weight % to about 15 weight %, based on the total weight of the composition. In yet another embodiment, the cleansing actives are present in an amount of from about 0.1 weight % to about 10 weight %, based on the total weight of the composition.

The extracts according to this disclosure may be used as anti-microbial and/or anti-fungal additives for cosmetic and/or pharmaceutical preparations used in skin and/or hair care. The anti-microbial and/or anti-fungal actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the anti-microbial and/or anti-fungal compositions of this disclosure in an amount effective for imparting the desired anti-microbial and/or anti-fungal effect in the desired time. A suitable effective amount of the anti-microbial and/or anti-fungal actives is about 0.001 weight % to about 25 weight %, based on the total weight of the anti-microbial and/or anti-fungal composition. In an embodiment, the anti-microbial and/or anti-fungal actives are present in an amount of from about 0.01 weight % to about 15 weight %, based on the total weight of the composition. In yet another embodiment, the anti-microbial and/or anti-fungal actives are present in an amount of from about 0.1 weight % to about 10 weight %, based on the total weight of the composition.

In an embodiment, the anti-microbial and/or anti-fungal additives can be used in cleansers, for example, hair, skin, body, and/or hand cleansers, to impart anti-microbial and/or anti-fungal properties to the cleanser. Such cleansers can be useful for removing body odor.

The extracts according to this disclosure may be used as additives in hair color applications. The additive actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the hair color compositions of this disclosure in an amount effective for imparting the desired hair coloring effect in the desired time. A suitable effective amount of the additive actives is about 0.001 weight % to about 25 weight %, based on the total weight of the hair coloring composition. In an embodiment, the additive actives are present in an amount of from about 0.01 weight % to about 15 weight %, based on the total weight of the composition. In yet another embodiment, the additive actives are present in an amount of from about 0.1 weight % to about 10 weight %, based on the total weight of the composition.

The cosmeceutical compositions of this disclosure are typically used in topical form. The topical form can be a solution, emulsion, serum, skin and/or hair cleanser, body wash, body scrub, bar soap, liquid soap, shampoo lather, deodorant, skin and/or hair care preparation, foam, mousse, cream, lotion, pomade, balm, stick, gel, pump spray, aerosol spray, and combinations thereof. Among other applications, the deodorants are useful for removing/controlling body odor.

In an illustrative embodiment, the cosmeceutical compositions of this disclosure are used in foams in personal care applications such as soaps, shampoos, skin cleansers, bubble bath, shaving soaps, oral products, and the like. The cosmeceutical compositions can impart desired foaming, emulsifying, cleansing, dispersing, and/or skin soothing properties.

The compositions of this disclosure comprise a “cosmeceutically acceptable carrier” to act as a diluant, dispersant or carrier for the ingredients, so as to facilitate its distribution and uptake when the composition is applied to the skin and/or hair. Vehicles other than or in addition to water can include liquid or solid emollients, solvents, humectants, thickeners, powders, and perfumes.

The cosmeceutically acceptable carrier will usually form from 0.001% to 99.9%, preferably from 25% to 80% by weight of the composition, and can, in the absence of other cosmeceutical adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase or conversely an aqueous phase in a fatty phase; or suspensions or emulsions of smooth, semi-solid or solid consistency of the cream or gel type. These compositions are formulated according to the conventional techniques known in the art.

When the compositions of this disclosure are formulated as an emulsion, the proportion of the fatty phase may range from 5% to 80% by weight, and preferably from 5% to 50% by weight, relative to the total weight of the composition. Oils, emulsifiers and co-emulsifiers incorporated in the composition in emulsion form are selected from among those used conventionally in the cosmetic or dermatological field. The emulsifier and coemulsifier may be present in the composition at a proportion ranging from 0.3% to 30% by weight, and preferably from 0.5% to 20% by weight, relative to the total weight of the composition.

When the compositions of this disclosure are formulated as an oily solution or gel, the fatty phase may constitute more than about 50%, more than about 75%, or even more than about 90% of the total weight of the composition.

The extracts according to this disclosure may be used in cosmetic and/or pharmaceutical preparations such as, for example, foam baths, shower baths, creams, gels, lotions, alcoholic and aqueous/alcoholic solutions, emulsions, wax/fat compounds, powders or ointments. These preparations may also contain surfactants, oil components, emulsifiers, pearlizing waxes, consistency factors, thickeners, superfatting agents, stabilizers, polymers, silicone compounds, fats, waxes, lecithins, phospholipids, biogenic agents, odor absorbers, film formers, swelling agents, hydrotropes, solubilizers, preservatives, perfume oils, dyes and the like, as further auxiliaries and additives.

Suitable surfactants include anionic, nonionic, cationic and/or amphoteric or zwitterionic surfactants which may be present in the preparations in quantities of normally about 1 to 70% by weight, preferably 5 to 50% by weight and more preferably 10 to 30% by weight. The surfactants are conventional materials known in the art.

Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isothionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides.

Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines.

Suitable oil components are, for example, alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate.

Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially dioctyl malate, esters of linear and/or branched fatty acids with polyhydric alcohols (for example propylene glycol, dimer diol or trimer triol), triglycerides based on C₆₋₁₀ fatty acids, liquid mono-, di- and triglyceride mixtures based on C₆₋₁₈ fatty acids, esters of C₆₋₂₂ fatty alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, carbonates, esters of benzoic acid with linear and/or branched C₆₋₂₂ alcohols, linear or branched, symmetrical or nonsymmetrical dialkyl ethers containing 6 to 22 carbon atoms per alkyl group, ring opening products of epoxidized fatty acid esters with polyols, silicone oils (cyclomethicone, silicon methicone types, etc.) and/or aliphatic or naphthenic hydrocarbons, for example squalane, squalene or dialkyl cyclohexanes.

The oil components may be present in the compositions of this disclosure in quantities of normally about 1 to about 90% by weight or greater, preferably about 5 to about 75% by weight and more preferably about 10 to about 50% by weight. The oil components are conventional materials known in the art.

Suitable emulsifiers include, for example, nonionic surfactants from at least one of the following groups: products of the addition of 2 to 30 moles of ethylene oxide and/or 0 to 5 moles of propylene oxide onto linear C₈₋₂₂ fatty alcohols, C₁₂₋₂₂ fatty acids and alkyl phenols containing 8 to 15 carbon atoms in the alkyl group and alkylamines containing 8 to 22 carbon atoms in the alkyl group; alkyl and/or alkenyl oligoglycosides containing 8 to 22 carbon atoms in the alkyl group and ethoxylated analogs thereof; adducts of 1 to 15 moles of ethylene oxide with castor oil and/or hydrogenated castor oil; adducts of 15 to 60 moles of ethylene oxide with castor oil and/or hydrogenated castor oil; partial esters of glycerol and/or sorbitan with unsaturated, linear or saturated, branched fatty acids containing 12 to 22 carbon atoms and/or hydroxycarboxylic acids containing 3 to 18 carbon atoms and adducts thereof with 1 to 30 moles of ethylene oxide; partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and adducts thereof with 1 to 30 moles of ethylene oxide; mixed esters of pentaerythritol, fatty acids, citric acid and fatty alcohol and/or mixed esters of fatty acids containing 6 to 22 carbon atoms, methyl glucose and polyols, preferably glycerol or polyglycerol, mono-, di- and trialkyl phosphates and mono-, di- and/or tri-PEG-alkyl phosphates and salts thereof, wool wax alcohols, polysiloxane/polyalkyl/polyether copolymers and corresponding derivatives, block copolymers, for example polyethylene glycol-30 dipolyhydroxystearate; polymer emulsifiers; polyalkylene glycols and glycerol carbonate.

The addition products of ethylene oxide and/or propylene oxide with fatty alcohols, fatty acids, alkylphenols or with castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of adducts of ethylene oxide with glycerol may be useful.

Alkyl and/or alkenyl oligoglycosides can be useful. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 moles of ethylene oxide with the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 moles of ethylene oxide with the sorbitan esters mentioned are also suitable.

Typical examples of suitable polyglycerol esters are polyglyceryl-2 dipolyhydroxystearate, polyglycerin-3-diisostearate, polyglyceryl-4 isostearate, polyglyceryl-3 oleate, diisostearoyl polyglyceryl-3 diisostearate, polyglyceryl-3 methylglucose distearate, polyglyceryl-3 beeswax, polyglyceryl-4 caprate, polyglyceryl-3 cetyl ether, polyglyceryl-3 distearate and polyglyceryl polyricinoleate, polyglyceryl dimerate isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 moles of ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethylimidazolines containing 8 to 18 carbon atoms in the alkyl or acyl group and cocoacylaminoethyl hydroxyethyl carboxymethyl glycinate. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group.

Cationic surfactants are also suitable emulsifiers, those of the esterquat type, preferably methyl-quaternized difatty acid triethanolamine ester salts, being preferred.

The emulsifiers may be present in the compositions of this disclosure in quantities of normally about 0.01 to about 40% by weight, preferably about 0.5 to about 25% by weight, and more preferably about 0.5 to about 20% by weight. The emulsifiers are conventional materials known in the art.

Typical examples of fats include glycerides, i.e. solid or liquid, vegetable or animal products which consist essentially of mixed glycerol esters of higher fatty acids. Suitable waxes are natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known in the art as glycerophospholipids which are formed from fatty acids, glycerol, phosphoric acid and choline by esterification. Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycero-phosphates) which are normally classed as fats. Sphingosines and sphingolipids are also suitable.

The fats may be present in the compositions of this disclosure in quantities of normally about 0.01 to about 40% by weight, preferably about 0.5 to about 25% by weight, and more preferably about 0.5 to about 20% by weight. The fats are conventional materials known in the art.

Suitable pearlescing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxy-substituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

The pearlescing waxes may be present in the compositions of this disclosure in quantities of normally about 0.01 to about 20% by weight, preferably about 0.5 to about 15% by weight, and more preferably about 0.5 to about 10% by weight. The pearlescing waxes are conventional materials known in the art.

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used.

The consistency factors may be present in the compositions of this disclosure in quantities of normally about 0.01 to about 20% by weight, preferably about 0.5 to about 15% by weight, and more preferably about 0.5 to about 10% by weight. The consistency factors are conventional materials known in the art.

Suitable thickeners are, for example, hydrophilic silicas, polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates; polyacrylamides, polyvinyl alcohol and polyvinyl pyrrolidone, surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

The thickeners may be present in the compositions of this disclosure in quantities of normally about 0.01 to about 20% by weight, preferably about 0.5 to about 15% by weight, and more preferably about 0.5 to about 10% by weight. The thickeners are conventional materials known in the art.

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

The superfatting agents may be present in the compositions of this disclosure in quantities of normally about 0.01 to about 20% by weight, preferably about 0.5 to about 15% by weight, and more preferably about 0.5 to about 10% by weight. The superfatting agents are conventional materials known in the art.

Metal salts of fatty acids such as, for example, magnesium, aluminum and/or zinc stearate or ricinoleate may be used as stabilizers.

The stabilizers may be present in the compositions of this disclosure in quantities of normally about 0.01 to about 20% by weight, preferably about 0.5 to about 15% by weight, and more preferably about 0.5 to about 10% by weight. The stabilizers are conventional materials known in the art.

Suitable cationic polymers are, for example, cationic cellulose derivatives such as, for example, quaternized hydroxyethyl cellulose, cationic starch, copolymers of diallyl ammonium salts and acrylamides, quaternized vinyl pyrrolidone/vinylimidazole polymers, condensation products of polyglycols and amines, quaternized collagen polypeptides such as, for example, lauryidimonium hydroxypropyl hydrolyzed collagen, quaternized wheat polypeptides, polyethyleneimine, cationic silicone polymers such as, for example, amodimethicone, copolymers of adipic acid and dimethylamino-hydroxypropyl diethylenetriamine, copolymers of acrylic acid with dimethyl diallyl ammonium chloride, polyaminopolyamides and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum, and quaternized ammonium salt polymers.

The cationic polymers may be present in the compositions of this disclosure in quantities of normally about 0.01 to about 20% by weight, preferably about 0.5 to about 15% by weight, and more preferably about 0.5 to about 10% by weight. The cationic polymers are conventional materials known in the art.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

The anionic, zwitterionic, amphoteric and nonionic polymers may be present in the compositions of this disclosure in quantities of normally about 0.01 to about 20% by weight, preferably about 0.5 to about 15% by weight, and more preferably about 0.5 to about 10% by weight. The anionic, zwitterionic, amphoteric and nonionic polymers are conventional materials known in the art.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

The silicone compounds may be present in the compositions of this disclosure in quantities of normally about 0.01 to about 20% by weight, preferably about 0.5 to about 15% by weight, and more preferably about 0.5 to about 10% by weight. The silicone compounds are conventional materials known in the art.

In the context of this disclosure, biogenic agents are, for example, tocopherol, tocopherol acetate, tocopherol palmitate, ascorbic acid, deoxyribonucleic acid, retinol, bisabolol, allantoin, phytantriol, panthenol, amino acids, ceramides, pseudoceramides, essential oils, other plant extracts and vitamin complexes.

The biogenic agents may be present in the compositions of this disclosure in quantities of normally about 0.01 to about 20% by weight, preferably about 0.5 to about 15% by weight, and more preferably about 0.5 to about 10% by weight. The biogenic agents are conventional materials known in the art.

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. They contain, for example, a complex zinc salt of ricinoleic acid or special perfumes of largely neutral odor such as, for example, extracts of ladanum or styrax or certain abietic acid derivatives as their principal component. Odor maskers are perfumes or perfume oils which, besides their odor-masking function, impart their particular perfume note to the cosmeceutical product. Suitable perfume oils are, for example, mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type.

Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexyl-cinnamaldehyde, geraniol, benzyl acetone, cyclamen aldehyde, linalool, boisambrene forte, ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, evernyl, iraldein γ, phenylacetic acid, geranyl acetate, benzyl acetate, rose oxide, romilat, irotyl and floramat.

The odor absorbers may be present in the compositions of this disclosure in quantities of normally about 0.01 to about 20% by weight, preferably about 0.5 to about 15% by weight, and more preferably about 0.5 to about 10% by weight. The odor absorbers are conventional materials known in the art.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

The water soluble additives may be present in the compositions of this disclosure in quantities of normally about 0.01 to about 20% by weight, preferably about 0.5 to about 15% by weight, and more preferably about 0.5 to about 10% by weight. The water-soluble additives are conventional materials known in the art.

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

The film formers may be present in the compositions of this disclosure in quantities of normally about 0.01 to about 20% by weight, preferably about 0.5 to about 15% by weight, and more preferably about 0.5 to about 10% by weight. The film formers are conventional materials known in the art.

Suitable swelling agents for aqueous phases are montmorillonites, clay minerals, pemulen and alkyl-modified carbopol types.

The swelling agents may be present in the compositions of this disclosure in quantities of normally about 0.01 to about 20% by weight, preferably about 0.5 to about 15% by weight, and more preferably about 0.5 to about 10% by weight. The swelling agents are conventional materials known in the art.

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are glycerol; alkylene glycols such as, for example, ethylene glycol, diethylene glycol, propylene glycol, butylene glycol, hexylene glycol and polyethylene glycols with an average molecular weight of 100 to 1000 dalton; technical oligoglycerol mixtures with a degree of self-condensation of 1.5 to 10 such as, for example, technical diglycerol mixtures with a diglycerol content of 40 to 50% by weight; methylol compounds such as, in particular, trimethylol ethane, trimethylol propane, trimethylol butane, pentaerythritol and dipentaerythritol; lower alkyl glucosides, particularly those containing 1 to 8 carbon atoms in the alkyl group, for example methyl and butyl glucoside; sugar alcohols containing 5 to 12 carbon atoms, for example sorbitol or mannitol, sugars containing 5 to 12 carbon atoms, for example glucose or sucrose; amino sugars, for example glucamine; dialcoholamines, such as diethanolamine or 2-aminopropane-1,3-diol.

The hydrotropes may be present in the compositions of this disclosure in quantities of normally about 0.01 to about 20% by weight, preferably about 0.5 to about 15% by weight, and more preferably about 0.5 to about 10% by weight. The hydrotropes are conventional materials known in the art.

Suitable preservatives are, for example, phenoxyethanol, formaldehyde solution, parabens, pentanediol or sorbic acid.

The preservatives may be present in the compositions of this disclosure in quantities of normally about 0.01 to about 20% by weight, preferably about 0.5 to about 15% by weight, and more preferably about 0.5 to about 10% by weight. The preservatives are conventional materials known in the art.

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used.

Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance.

Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, boisambrene forte, ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, iraldein γ, phenylacetic acid, geranyl acetate, benzyl acetate, rose oxide, romillat, irotyl and floramat.

The perfume oils may be present in the compositions of this disclosure in quantities of normally about 0.01 to about 20% by weight, preferably about 0.5 to about 15% by weight, and more preferably about 0.5 to about 10% by weight. The perfume oils are conventional materials known in the art.

Suitable dyestuffs and colorants are any of the substances suitable and approved for cosmetic and/or pharmaceutical purposes. The dyestuffs and colorants are conventional materials known in the art. The dyestuffs and colorants are normally used in concentrations of 0.001 to 0.1% by weight, based on the mixture as a whole.

The compositions of this disclosure can also include one or more other plant extracts having desired cosmeceutical or other properties. Illustrative of other plant extracts include, for example, Withania somnifera, Silybum marianum, Curcuma longa, Camellia sinensis, and the like.

The other plant extracts may be present in the compositions of this disclosure in quantities of normally about 0.001 to about 20% by weight, preferably about 0.01 to about 15% by weight, and more preferably about 0.1 to about 10% by weight. The other plant extracts are known in the art.

Oral hygiene involves keeping the mouth and teeth clean to prevent dental problems and bad breath. Mouthwashes and mouth rinses are products used to enhance oral hygiene. Active ingredients in commercial brands of mouthwash can include thymol, eucalyptol, hexetidine, methyl salicylate, menthol, chlorhexidine gluconate, benzalkonium chloride, cetylpyridinium chloride, methylparaben, hydrogen peroxide, domiphen bromide and sometimes fluoride, enzymes and calcium. Ingredients also include water, sweeteners such as sorbitol, sucralose, sodium saccharine, and xylitol (which doubles as a bacterial inhibitor). Sometimes alcohol is added, as a carrier for flavor, and to contribute an anti-bacterial effect. Commercial mouthwashes usually contain a preservative such as sodium benzoate to preserve freshness once the container has been opened. Many brands contain odor-elimination agents such as oxidizers, as well as odor-preventing agents such as zinc ion to keep future bad breath from developing.

The extracts according to this disclosure may be used as cleaning and/or deodoring additives for mouthwashes, mouth rinses, and the like. The cleaning and/or deodoring actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the mouthwashes, mouth rinses, and the like, in an amount effective for imparting the desired cleaning and/or deodoring effect in the desired time. A suitable effective amount of the cleaning and/or deodoring actives is about 0.001 weight % to about 25 weight %, based on the total weight of the composition. In an embodiment, the cleaning and/or deodoring actives are present in an amount of from about 0.01 weight % to about 15 weight %, based on the total weight of the composition. In yet another embodiment, the cleaning and/or deodoring actives are present in an amount of from about 0.1 weight % to about 10 weight %, based on the total weight of the composition.

This disclosure also relates to a process for preparing an extract of the Bacopa plant, Centella plant, Jatropha plant, Aegle plant, Terminalia plant, Phyllanthus plant, Spilanthes plant, and/or mixtures thereof, in which solvents or mixtures of solvents selected from the group consisting of distilled or nondistilled water, low molecular weight alcohols, esters or hydrocarbons are used for extraction of the plant.

It has been found that selective Bacopa natural products, Centella natural products, Jatropha natural products, Aegle natural products, Terminalia natural products, Phyllanthus natural products, Spilanthes natural products, and/or mixtures thereof, have the exceptional quality to absorb the UV light completely, for example between 190 and 400 nm. These Bacopa natural products, Centella natural products, Jatropha natural products, Aegle natural products, Terminalia natural products, Phyllanthus natural products, Spilanthes natural products, and/or mixtures thereof, not only can absorb UV lights, but also can scavenge free radicals efficiently. For example, the free radical scavenging capacity of these Bacopa products, Centella products, Jatropha products, Aegle products, Terminalia products, Phyllanthus products, Spilanthes products, and/or mixtures thereof, can be more than 100 times superior to the presently available antioxidants. Furthermore, a few of these products found to have a strong moisturizing as well as sunless tanning and sunscreen properties. The presence of these properties makes these Bacopa products, Centella products, Jatropha products, Aegle products, Terminalia products, Phyllanthus products, Spilanthes products, and/or mixtures thereof, a perfect environmentally benign candidate for the cosmeceuticals and UV additive applications.

It has further been found that Bacopa natural products, Centella natural products, Jatropha natural products, Aegle natural products, Terminalia natural products, Phyllanthus natural products, Spilanthes natural products, and/or mixtures thereof, have the capability to absorb both UVA and UVB radiation in addition to having the free radical scavenging capacity. Many of the Bacopa natural products, Centella natural products, Jatropha natural products, Aegle natural products, Terminalia natural products, Phyllanthus natural products, Spilanthes natural products, and/or mixtures thereof, are water soluble, therefore, it is possible to formulate oil free sunscreens in addition to fulfilling all other requirements.

It has also been found that the Bacopa natural products, Centella natural products, Jatropha natural products, Aegle natural products, Terminalia natural products, Phyllanthus natural products, Spilanthes natural products, and/or mixtures thereof, have strong antioxidant properties which have the capability to terminate free radical chain reactions in biological systems and therefore may provide additional health benefits to consumers. Severe oxidative stress, a result of an imbalance between antioxidative defense systems and the formation of reactive oxygen species including free radicals, can damage DNA, proteins, lipids and carbohydrate and may also alter intracellular signaling processes. The damage could contribute to cell injury and death, accelerate the aging process, and promote many diseases, such as cancer, cardiovascular diseases, diabetes, arthritis, Alzheimer's disease, Parkinson's disease, and free radical related diseases.

The Bacopa natural products, Centella natural products, Jatropha natural products, Aegle natural products, Terminalia natural products, Phyllanthus natural products, Spilanthes natural products, and/or mixtures thereof, of this disclosure can be utilized in many cosmeceutical applications. Preferred cosmeceutical applications include, for example, the following:

(i) sunscreen lotions; since the Bacopa extracts, Centella extracts, Jatropha extracts, Aegle extracts, Terminalia extracts, Phyllanthus extracts, Spilanthes extracts, and/or mixtures thereof, have the capability to protect skin and/or hair against three types of ultraviolet radiations, i.e., ultraviolet-B (UV-B) and ultraviolet-A (UV-A) that reach earth's atmosphere from the sun and ultraviolet-C (UV-C) that does not reach the earth atmosphere, the Bacopa extracts, Centella extracts, Jatropha extracts, Aegle extracts, Terminalia extracts, Phyllanthus extracts, Spilanthes extracts, and/or mixtures thereof, can be used to make sun protecting lotions;

(ii) anti aging or anti wrinkling creams; the Bacopa extract, Centella extract, Jatropha extract, Aegle extract, Terminalia extract, Phyllanthus extract, Spilanthes extract, and/or mixtures thereof, products have natural antioxidant properties in addition to having the capacity to scavenge radicals causing damage to human cells; these properties are known to decelerate the aging process;

(iii) sunless tanning agents; the Bacopa extract, Centella extract, Jatropha extract, Aegle extract, Terminalia extract, Phyllanthus extract, Spilanthes extract, and/or mixtures thereof, products have the capacity to tan skin in absence of sunlight; and

(iv) moisturizers; the Bacopa extract, Centella extract, Jatropha extract, Aegle extract, Terminalia extract, Phyllanthus extract, Spilanthes extract, and/or mixtures thereof, products have the potential to protect the skin from the water loss which ultimately reduces the risks of many skin diseases.

These Bacopa plant based products, Centella plant based products, Jatropha plant based products, Aegle plant based products, Terminalia plant based products, Phyllanthus plant based products, Spilanthes plant based products, and/or mixtures thereof, not only have the potential to replace currently used unhealthy synthetic chemicals for cosmeceuticals and other UV additive applications, but also provide additional benefits to the health of consumers, and help to prevent further synthetic chemical pollution in the environment.

In addition, the Bacopa natural products, Centella natural products, Jatropha natural products, Aegle natural products, Terminalia natural products, Phyllanthus natural products, Spilanthes natural products, and/or mixtures thereof, of this disclosure can be utilized in many pharmaceutical applications. Illustrative pharmaceutical applications include, for example, the treatment of cancer, diabetes, heart disease, arthritis, Alzheimer's disease, Parkinson's disease, free radical related diseases, and the like.

The Bacopa plant based products, Centella plant based products, Jatropha plant based products, Aegle plant based products, Terminalia plant based products, Phyllanthus plant based products, Spilanthes plant based products, and/or mixtures thereof, can also be utilized in other applications. For example, the Bacopa plant based products, Centella plant based products, Jatropha plant based products, Aegle plant based products, Terminalia plant based products, Phyllanthus plant based products, Spilanthes plant based products, and/or mixtures thereof, can be used as stabilizers (e.g., for coatings, paints, polymers, plastics, emulsions, and the like), surfactants (e.g., for oil recovery, soil cleaning, mineral separation, agrochemical applications, and the like), preservatives (e.g., food additives), treatment agents (e.g., additives), dispersants, chelating agents, emulsifiers, surface modifying agents, foaming/frothing agents, surface active agents, cleansing/degreasing agents (e.g., laundry and dishwashing detergents), and rheology modifying agents.

The Bacopa plant based products, Centella plant based products, Jatropha plant based products, Aegle plant based products, Terminalia plant based products, Phyllanthus plant based products, Spilanthes plant based products, and/or mixtures thereof, include extracts that can be utilized in various applications. Illustrative extracts of this disclosure include, for example, stabilizer active extracts of a Bacopa plant, a Centella plant, a Jatropha plant, a Aegle plant, a Terminalia plant, a Phyllanthus plant, a Spilanthes plant, or mixtures thereof; surfactant active extracts a Bacopa plant, a Centella plant, a Jatropha plant, an Aegle plant, a Terminalia plant, a Phyllanthus plant, a Spilanthes plant, or mixtures thereof; preservative active extracts a Bacopa plant, a Centella plant, a Jatropha plant, a Aegle plant, a Terminalia plant, a Phyllanthus plant, a Spilanthes plant, or mixtures thereof; treatment active extracts a Bacopa plant, a Centella plant, a Jatropha plant, a Aegle plant, a Terminalia plant, a Phyllanthus plant, a Spilanthes plant, or mixtures thereof; dispersant active extracts of a Bacopa plant, a Centella plant, a Jatropha plant, a Aegle plant, a Terminalia plant, a Phyllanthus plant, a Spilanthes plant, or mixtures thereof; chelating active extracts of a Bacopa plant, a Centella plant, a Jatropha plant, a Aegle plant, a Terminalia plant, a Phyllanthus plant, a Spilanthes plant, or mixtures thereof; emulsifier active extracts of a Bacopa plant, a Centella plant, a Jatropha plant, a Aegle plant, a Terminalia plant, a Phyllanthus plant, a Spilanthes plant, or mixtures thereof; surface modifying active extracts of a Bacopa plant, a Centella plant, a Jatropha plant, a Aegle plant, a Terminalia plant, a Phyllanthus plant, a Spilanthes plant, or mixtures thereof; foaming/frothing active extracts of a Bacopa plant, a Centella plant, a Jatropha plant, a Aegle plant, a Terminalia plant, a Phyllanthus plant, a Spilanthes plant, or mixtures thereof; surface active agent active extracts of a Bacopa plant, a Centella plant, a Jatropha plant, a Aegle plant, a Terminalia plant, a Phyllanthus plant, a Spilanthes plant, or mixtures thereof; cleansing/degreasing active extracts of a Bacopa plant, a Centella plant, a Jatropha plant, a Aegle plant, a Terminalia plant, a Phyllanthus plant, a Spilanthes plant, or mixtures thereof; and rheology modifying active extracts of a Bacopa plant, a Centella plant, a Jatropha plant, a Aegle plant, a Terminalia plant, a Phyllanthus plant, a Spilanthes plant, or mixtures thereof.

The stabilizer active extracts are useful, for example, as stabilizers in coatings, paints, polymers, plastics and emulsions. The surfactant active extracts are useful, for example, in oil recovery, soil cleaning, mineral separation, agrochemicals, detergents, food product preparation, nutraceuticals, coating, printing inks, electronic printing, microelectronics, magnetic recording, high technology electronic ceramics, batteries, and particle dispersion. The preservative active extracts are useful, for example, as food preservatives. The treatment active extracts are useful, for example, in fabric and fiber care, e.g., UV protection. The dispersant active extracts are useful, for example, as dispersants in oil recovery, oil cleanup, food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, biotechnology, particle dispersion, laundry, detergents, cleaning, washing (e.g., automobiles, trucks, trains, airplanes, and the like), and degreasing. The chelating active extracts are useful, for example, in metal extraction and washing (e.g., automobiles, trucks, trains, airplanes, and the like). The emulsifier active extracts are useful, for example, in oil recovery, oil cleanup, food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, and biotechnology.

The surface modifying active extracts are useful, for example, in food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, electronic printing, microelectronics, magnetic recording, high technology electronic ceramics, biotechnology, separation techniques, and particle dispersion. The foaming/frothing active extracts are useful, for example, in fire fighting and oil recovery. The surface active agent active extracts are useful, for example, in oil recovery, soil cleaning, mineral separation, agrochemicals, detergents, food product preparation, nutraceuticals, coating, printing inks, electronic printing, microelectronics, magnetic recording, high technology electronic ceramics, batteries, and particle dispersion. The cleansing/degreasing active extracts are useful, for example, in food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, electronic printing, microelectronics, magnetic recording, high technology electronic ceramics, biotechnology, laundry, detergents, and washing (e.g., automobiles, trucks, trains, airplanes, and the like). The rheology modifying active extracts are useful, for example, in thickening and thinning of fluids, suspensions, polymers, foods and additives, bodily fluids (e.g., blood) and other biological materials.

Illustrative compositions of this disclosure containing at least one of a Bacopa plant extract, a Centella plant extract, a Jatropha plant extract, an Aegle plant extract, a Terminalia plant extract, a Phyllanthus plant extract, a Spilanthes plant extract, or mixtures thereof, include for example, stabilizer compositions, surfactant compositions, preservative compositions, treatment compositions, dispersant compositions, chelating compositions, emulsifier compositions, surface modifying compositions, foaming/frothing compositions, surface active agent compositions, cleansing/degreasing compositions, and rheology modifying compositions. The compositions are useful in the various applications described above.

The extracts according to this disclosure may be used as food additives, e.g., preservatives. The food preservative actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the food preservative compositions of this disclosure in an amount effective for imparting the desired preservative effect in the desired time. A suitable effective amount of the preservative actives is about 0.001 weight % to about 25 weight %, based on the total weight of the food preservative composition. In an embodiment, the preservative actives are present in an amount of from about 0.01 weight % to about 15 weight %, based on the total weight of the composition. In yet another embodiment, the preservative actives are present in an amount of from about 0.1 weight % to about 10 weight %, based on the total weight of the composition.

Coatings, paints, polymers, plastics, emulsions, and the like, can have, for example, a light stabilizer (i.e., a Bacopa plant extract, Centella plant extract, Jatropha plant extract, Aegle plant extract, Terminalia plant extract, Phyllanthus plant extract, Spilanthes plant extract, and/or mixtures thereof) in combination with a UV absorber for optimal protection of coatings and plastics. The ratio of UV absorber and stabilizer depends on the concentration of pigments used in the materials, e.g., paint. Clear coatings require higher amounts of UV absorber, whereas opaque pigmented coatings require higher amounts of stabilizer.

The extracts according to this disclosure may be used as stabilizers, e.g., light stabilizers, for coatings, paints, polymers, plastics, emulsions, and the like. The stabilizer actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the coatings, paints, polymers, plastics, emulsions, and the like, in an amount effective for imparting the desired stabilizer effect in the desired time. A suitable effective amount of the stabilizer actives is about 0.001 weight % to about 25 weight %, based on the total weight of the composition. In an embodiment, the stabilizer actives are present in an amount of from about 0.01 weight % to about 15 weight %, based on the total weight of the composition. In yet another embodiment, the stabilizer actives are present in an amount of from about 0.1 weight % to about 10 weight %, based on the total weight of the composition.

Oil recovery, e.g., enhanced oil recovery, consists of injecting a displacing fluid into injection wells in order to displace the oil and gas in a reservoir towards producing wells. An enhanced oil recovery technique involves chemical flooding in which an alkaline-surfactant-polymer formulation is injected into the reservoir. The polymer is used to improve the sweep efficiency of the invading fluid by changing the mobility ratio between the invading fluid versus the displaced fluid. The surfactant is present to change the wettability of the formation rock if necessary and to reduce the interfacial tension.

The extracts, e.g., isolated molecules from the extracts, according to this disclosure may be used as surfactants in oil recovery formulations. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the oil recovery formulations in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition.

The extracts, e.g., isolated molecules from the extracts, according to this disclosure may also be used for foaming in oil recovery applications as well as firefighting applications. The extracts exhibit desired foaming properties.

Various oil cleanup methods can be employed for cleaning oil spills. Oil spills result from the release of crude oil into water, for example, from oil supertankers, underwater oil pipelines, offshore oil drilling rigs, coastal oil storage facilities and oil refineries. One method involves the use of dispersants to break up the oil and speed its natural biodegradation. Dispersants act by reducing the surface tension that stops oil and water from mixing. Small droplets of oil are then formed, which helps promote rapid dilution of the oil by water movements. The formation of droplets also increases the oil surface area, thus increasing the exposure to natural evaporation and bacterial action.

The extracts, e.g., isolated molecules from the extracts, according to this disclosure may be used as dispersants or emulsifying agents in oil cleanup formulations. The dispersant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the oil cleanup formulations in an amount effective for imparting the desired dispersant or emulsifying effect in the desired time. A suitable effective amount of the dispersant actives is about 0.001 weight % to about 25 weight %, based on the total weight of the composition. In an embodiment, the dispersant actives are present in an amount of from about 0.01 weight % to about 15 weight %, based on the total weight of the composition. In yet another embodiment, the dispersant actives are present in an amount of from about 0.1 weight % to about 10 weight %, based on the total weight of the composition.

The extracts of this disclosure may also impart dispersing, surface modifying and surface active properties to the oil cleanup formulations. In addition to oil cleanup, the extracts of this disclosure may be useful for particle dispersion applications in which the dispersant actives impart dispersing, surface modifying and surface active properties to the particle dispersion formulations.

Soil cleaning processes are used in for the treatment of contaminated soil, for example, oil contaminated soil. Soil cleaning or washing is a water-based process for scrubbing soils ex situ to remove contaminants. Contaminants sorbed onto fine soil particles are separated from bulk soil in an aqueous-based system on the basis of particle size. The soil cleaning formulation includes wash water that may be augmented with a basic leaching agent, surfactant, pH adjustment, or chelating agent to help remove organics and heavy metals.

The extracts according to this disclosure may be used as surfactants in soil cleaning formulations. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the soil cleaning formulations in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition.

Mineral separation is important in the mining industry, for example, in the separation of valuable minerals from complex ores. Surface characteristics of minerals are often complex due to changes during particle preparation as well as interactions with the surrounding environment. The key to their separation lies in manipulation of the surface characteristics by treatment with selected reagents. Controlled adsorption of surfactants, polymers or a combination of both can modify the surface of the mineral to achieve the desired separation results.

The extracts according to this disclosure may be used as surfactants in mineral separation formulations. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the mineral separation formulations in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition.

In addition to mineral separation, the extracts of this disclosure may be useful in other separation techniques. The extracts of this disclosure may also impart surface modification, foam stabilization and frothing properties to formulations used in other separation techniques.

Chelating agents are important in a variety of applications, for example, metal extraction, textile dyeing, water softening, enzyme deactivation, bacteriocides, and the like. The chelating agents can combine with metal ions and remove them from their sphere of action. For example, they can be used in food manufacture to remove traces of metal ions which might otherwise cause foods to deteriorate, and clinically to reduce absorption of a mineral, or to increase its excretion, e.g., citrates, tartrates and phosphates.

The extracts according to this disclosure may be used as chelating agents in the various application formulations. The chelating agents, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the formulations in an amount effective for imparting the desired chelating effect in the desired time. A suitable effective amount of the chelating agents is about 0.001 weight % to about 25 weight %, based on the total weight of the composition. In an embodiment, the chelating agents are present in an amount of from about 0.01 weight % to about 15 weight %, based on the total weight of the composition. In yet another embodiment, the chelating agents are present in an amount of from about 0.1 weight % to about 10 weight %, based on the total weight of the composition.

Surface modifying processes are used to modify the surface of a material by bringing physical, chemical or biological characteristics different from the ones originally found on the surface of the material. Surface modification can involve using surface modifying formulations to alter a wide range of characteristics of the surface such as roughness, hydrophilicity, surface charge, surface energy, biocompatibility and reactivity.

The extracts according to this disclosure may be used as surface modifers in surface modifying formulations. The surface modifer actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the surface modifying formulations in an amount effective for imparting the desired surface modifying effect in the desired time. A suitable effective amount of the surface modifer actives is about 0.001 weight % to about 25 weight %, based on the total weight of the composition. In an embodiment, the surface modifer actives are present in an amount of from about 0.01 weight % to about 15 weight %, based on the total weight of the composition. In yet another embodiment, the surface modifer actives are present in an amount of from about 0.1 weight % to about 10 weight %, based on the total weight of the composition.

Rheology has applications in materials science engineering, human biology, pharmaceutics, and the like. Materials science is utilized in the production of many industrially important substances such as concrete and paint. Rheology has played an important role in the production of many products for use in both the industrial and military sectors.

The extracts according to this disclosure may be used as rheology modifers in various formulations such as paints. The rheology modifer actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the formulations in an amount effective for imparting the desired rheology modifying effect in the desired time. A suitable effective amount of the rheology modifer actives is about 0.001 weight % to about 25 weight %, based on the total weight of the composition. In an embodiment, the rheology modifer actives are present in an amount of from about 0.01 weight % to about 15 weight %, based on the total weight of the composition. In yet another embodiment, the rheology modifer actives are present in an amount of from about 0.1 weight % to about 10 weight %, based on the total weight of the composition.

Agrochemicals such as pesticides, animal food additives, veterinary drugs, and related compounds, have become integral to the production of large-scale agriculture. Agrochemicals deal with all aspects of pesticide and herbicide delivery systems, including emulsifiable concentrates, water dispersible granules, wettable powders, suspo-emulsions, micro-emulsions, capsule suspensions, and low aquatic toxicity adjuvants.

The extracts according to this disclosure may be used as surfactants in agrochemical formulations. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the agrochemical formulations in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition.

Emulsifiers are used to stabilize an emulsion by increasing its kinetic stability. Emulsifiers can be used for food, drug, cosmetics (both hair and skin care), agrochemicals, paints, paper making, printing, graphic arts, lithography, hydraulic fluid emulsions, fuels, lubricants, coatings and polymers, bioreactive materials, extraction media, oil recovery applications, and the like. The emulsifiers can exhibit, in addition to emulsifying properties, dispersing, cleansing and surface modifying properties.

The extracts according to this disclosure may be used as emulsifiers in emulsion formulations. The emulsifiers, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the emulsion formulations in an amount effective for imparting the desired emulsifying effect in the desired time. A suitable effective amount of the emulsifiers is about 0.001 weight % to about 25 weight %, based on the total weight of the composition. In an embodiment, the emulsifiers are present in an amount of from about 0.01 weight % to about 15 weight %, based on the total weight of the composition. In yet another embodiment, the emulsifiers are present in an amount of from about 0.1 weight % to about 10 weight %, based on the total weight of the composition.

Laundry detergent typically consists of (i) ionic and anionic surfactants which act as the detergent to remove the dirt from the clothes, (ii) perfume, (iii) phosphors which make clothes appear whiter. Laundry powders have anticaking agents to prevent the powder becoming one large lump in the presence of moisture. For liquid detergents, the bulk of the product is water; for concentrated liquids, somewhat less water, but still the product is mostly water. Detergents may have other additives such as bleaches and fabric softeners.

Dishwashing detergents are usually a highly foaming mixture of surfactants with low skin irritation, and are primarily used for washing of glasses, plates, cutlery, and cooking utensils. The reduced surface tension of dishwashing water, and increasing solubility of surfactant mixtures, allows the soap to run off the dishes in a dish rack and the remaining traces of dishwashing liquid can dry off fast and do not pose any health or taste problems.

The extracts according to this disclosure may be used as surfactants for detergents such as laundry detergents, dishwashing detergents, general household cleaning detergents, and the like. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the detergents in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition. The extracts of this disclosure can impart cleansing, dispersing, degreasing and chelating properties to the detergents.

In addition to surfactant properties, the extracts of this disclosure may impart other desired properties such as anti-bacterial, anti-viral and anti-fungal properties to the laundry detergents, dishwashing detergents, general household cleaning detergents, and the like. The extracts of this disclosure may also impart cleansing, dispersing, degreasing and chelating properties to the laundry detergents, dishwashing detergents, general household cleaning detergents, and the like.

In addition to laundry detergents, dishwashing detergents, and general household cleaning detergents, the extracts of this disclosure may be used in washing and cleansing formulations for heavy duty applications such as vehicle washing, truck washing, train washing, airplane washing, and the like. The extracts of this disclosure can impart cleansing, dispersing, degreasing and chelating properties to the washing formulations.

Dust control aims at the suppression of solid particles with diameters less than 500 micrometers. Dust in the airstream poses serious health problems to children, older people, and those with respiratory diseases. House dust can become airborne easily. Care should be exercised when removing dust to avoid causing the dust to become airborne. Dust in homes, offices, and other human environments can contain small amounts of plant pollen, animal and human hairs, textile fibers, paper fibers, minerals from outdoor soil, and many other materials that may be found in the local environment.

The extracts according to this disclosure may be used as dust controlling agents, e.g., surface modifying, chelating and surface active agents for controlling dust. The dust controlling actives, e.g., Bacopa plant extract, Centella plant extract, Jatropha plant extract, Aegle plant extract, Terminalia plant extract, Phyllanthus plant extract, Spilanthes plant extract, and/or mixtures thereof, are used in compositions in an amount effective for imparting the desired surface modifying, chelating and/or surface active effect in the desired time. A suitable effective amount of the dust controlling actives is about 0.001 weight % to about 25 weight %, based on the total weight of the composition. In an embodiment, the dust controlling actives are present in an amount of from about 0.01 weight % to about 15 weight %, based on the total weight of the composition. In yet another embodiment, the dust controlling actives are present in an amount of from about 0.1 weight % to about 10 weight %, based on the total weight of the composition.

The properties of many food products such as volume and texture are dependent upon the interaction of various ingredients during preparation thereof, and on the stabilization during subsequent baking, handling and storage of the food products. Surfactants can be used to stabilize food products such as butter, yogurt, cheese, ice cream and the like to keep the food products adequately dispersed and to control rheology.

The extracts, e.g., individual molecular compositions, according to this disclosure may be used as surfactants for food products and nutraceuticals. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the food products and nutraceuticals in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition. Mixtures of extracts may be particularly useful in this application.

As used herein, “nutraceuticals” mean food or food products that provide health and medical benefits, including the prevention and treatment of disease. Such products may range from isolated nutrients, dietary supplements, and specific diets to genetically engineered foods, herbal products, and processed foods such as cereals, soups and beverages.

The topical application of liquid compositions, e.g., coating and printing ink compositions, to surfaces to effect a desired change involve processes of controlling wetting, spreading, flow, leveling, foaming, detergency, and the like. Surfactants can be used to obtain desired properties.

The extracts according to this disclosure may be used as surfactants for coating and printing ink compositions. The surfactant actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the coating and printing ink compositions in an amount effective for imparting the desired surfactant effect in the desired time. A suitable effective amount of the surfactant actives is about 0.001 weight % to about 25 weight %, based on the total weight of the composition. In an embodiment, the surfactant actives are present in an amount of from about 0.01 weight % to about 15 weight %, based on the total weight of the composition. In yet another embodiment, the surfactant actives are present in an amount of from about 0.1 weight % to about 10 weight %, based on the total weight of the composition.

The extracts according to this disclosure may be used as additives in fabric and fiber care compositions to impart desired properties such as, for example, UV protection and fabric softening. The UV protection/fabric softening actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the compositions in an amount effective for imparting the desired UV protection/fabric softening effect in the desired time. A suitable effective amount of the UV protection/fabric softening actives is about 0.001 weight % to about 25 weight %, based on the total weight of the composition. In an embodiment, the UV protection/fabric softening actives are present in an amount of from about 0.01 weight % to about 15 weight %, based on the total weight of the composition. In yet another embodiment, the UV protection/fabric softening actives are present in an amount of from about 0.1 weight % to about 10 weight %, based on the total weight of the composition.

Biotechnology has applications in at least four major industrial areas, including health care (medical), crop production and agriculture, non food (industrial) uses of crops and other products (e.g., biodegradable plastics, vegetable oil and biofuels), and environmental uses. The extracts according to this disclosure may be used as additives in biotechnology formulations in the above areas to impart desired properties such as, for example, surface modification, emulsification, dispersion, detergent, UV absorption, antioxidant properties, and the like.

The actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the biotechnology formulations in an amount effective for imparting the desired surfactant effect in the desired time. A suitable effective amount of the actives is about 0.001 weight % to about 25 weight %, based on the total weight of the composition. In an embodiment, the actives are present in an amount of from about 0.01 weight % to about 15 weight %, based on the total weight of the composition. In yet another embodiment, the actives are present in an amount of from about 0.1 weight % to about 10 weight %, based on the total weight of the composition. Mixtures of extracts may be particularly useful in this application.

The extracts according to this disclosure may be used in compositions for electronic printing, microelectronics, magnetic recording, high technology electronic ceramics, advanced battery technology, and the like, to impart desired properties such as, for example, dispersing, surface modifying, and detergent/surface active agent properties. The actives, e.g., Bacopa plant extract, Centella plant extract, Jatropha plant extract, Aegle plant extract, Terminalia plant extract, Phyllanthus plant extract, Spilanthes plant extract, and/or mixtures thereof, are present in the compositions in an amount effective for imparting the desired effect in the desired time. A suitable effective amount of actives is about 0.001 weight % to about 25 weight %, based on the total weight of the composition. In an embodiment, the actives are present in an amount of from about 0.01 weight % to about 15 weight %, based on the total weight of the composition. In yet another embodiment, the actives are present in an amount of from about 0.1 weight % to about 10 weight %, based on the total weight of the composition.

Various modifications and variations of this disclosure will be obvious to a worker skilled in the art and it is to be understood that such modifications and variations are to be included within the purview of this application and the spirit and scope of the claims.

### Example 1

The active soluble components from Bacopa monniera (stems, leaves and roots) were air-dried and ground to form powder. 100 grams of the powder was extracted three times using water alone, ethanol alone, methanol and different ratio of water:methanol and water:ethanol solution at a temperature of 20-90° C. for a period of 24 hours. The supernatant was separated from the solid residue by filtration. The solvent in the supernatant was removed by evaporation at 50° C. under reduced pressure. The resulting suspension was then lyophilized to yield powder using the extract. The lyophilized powder was refrigerated and used for all the studies.

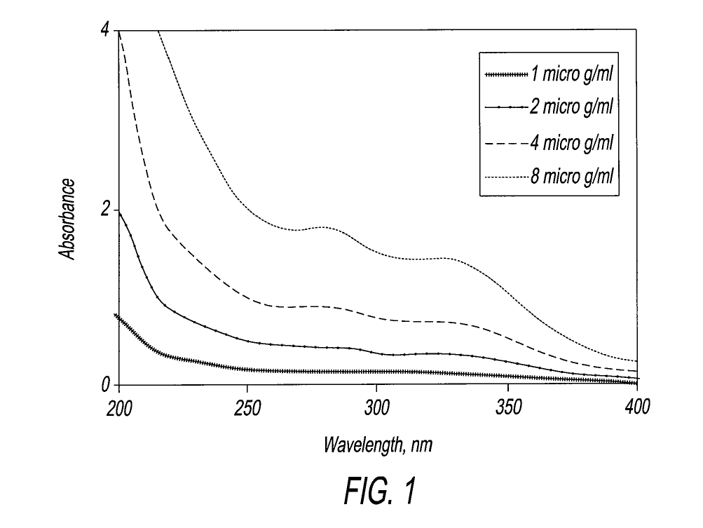

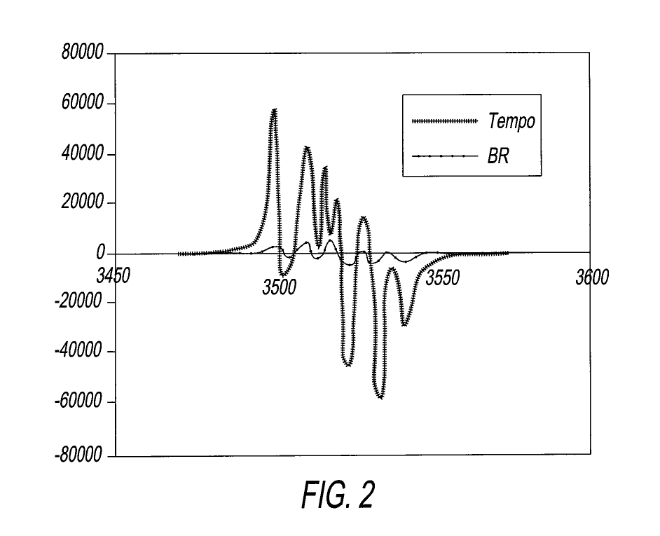

A known amount of the freeze dried sample was dissolved in triple distilled water and then absorbance of the solution was measured as a function of wavelength at different concentrations and illustrated in FIGS. 1 and 2. The Bacopa monniera plant extract has the exceptional quality to absorb the ultraviolet (UV) light completely, for example, between 190-400 nm.

FIG. 1 shows UV-visible spectra of Bacopa monniera plant extract at various different concentrations. It is evident from FIG. 1 that the Bacopa monniera extract has the capability to absorb light in the entire UV range, for example, from 190-400 nm.

### Example 2

It has also been demonstrated that these natural Bacopa monniera extract products not only can absorb UV lights, but also can scavenge free radicals efficiently. The free radical scavenging capacity of Bacopa monniera plant extract was evaluated using electron spin resonance (ESR) techniques. ESR is considered to be the least ambiguous method for the detection of free radicals. Free radical scavenging activity results are illustrated in FIG. 2.

The nitroxide free radical tetramethylpiperidinyl-1-oxy (Tempo) was used as a stable free radical. An alcohol solution of Tempo was mixed with Bacopa monniera plant extract for 2 minutes followed by ESR measurements. Model ESR spectra of Tempo in the absence and the presence of Bacopa monniera plant extract are illustrated in FIG. 2. 100 μg of Bacopa monniera plant extract resulted in almost disappearance of the Tempo free radical peaks.

FIG. 2 shows the free radical scavenging activity of Bacopa monniera plant extract determined by ESR, in particular, that 100 μg of plant extract can scavenge 1 mg of Tempo stable free radicals completely.

### Example 3

The free radical scavenging capacity of the Bacopa monniera extract product was evaluated using spectrophotometric and electron spin resonance (ESR) techniques. ESR is considered to be the least ambiguous method for the detection of free radicals.

2,2-Diphenyl-1-pierylhydrazyl (DPPH) was used as a stable free radical. An alcohol solution of DPPH was mixed with 100 μg of a Bacopa monniera extract product for 2 minutes and then ESR measurements were conducted. Quercetine was used as a positive control for scavenging the free radicals. A slight change in peak height was observed in the presence of 100 μg of quercetine, while 100 μg of the Bacopa monniera extract product resulted in the disappearance of free radical peak.

What is claimed is:

1. A cosmeceutically active extract selected from the group consisting of a Bacopa plant extract, a Centella plant extract, a Jatropha plant extract, an Aegle plant extract, a Terminalia plant extract, a Phyllanthus plant extract, a Spilanthes plant extract, and mixtures thereof.

2. A cosmeceutically active extract selected from the group consisting of a sunless tanning active extract of a Bacopa plant, a Centella plant, a Jatropha plant, an Aegle plant, a Terminalia plant, a Phyllanthus plant, a Spilanthes plant, or mixtures thereof; a sunscreen active extract of a Bacopa plant, a Centella plant, a Jatropha plant, an Aegle plant, a Terminalia plant, a Phyllanthus plant, a Spilanthes plant, or mixtures thereof; an ultraviolet radiation protective extract of a Bacopa plant, a Centella plant, a Jatropha plant, an Aegle plant, a Terminalia plant, a Phyllanthus plant, a Spilanthes plant, or mixtures thereof; an anti-aging active extract of a Bacopa plant, a Centella plant, a Jatropha plant, an Aegle plant, a Terminalia plant, a Phyllanthus plant, a Spilanthes plant, or mixtures thereof; an anti-wrinkle active extract of a Bacopa plant, a Centella plant, a Jatropha plant, an Aegle plant, a Terminalia plant, a Phyllanthus plant, a Spilanthes plant, or mixtures thereof; a moisturizing active extract of a Bacopa plant, a Centella plant, a Jatropha plant, an Aegle plant, a Terminalia plant, a Phyllanthus plant, a Spilanthes plant, or mixtures thereof; a skin soothing active extract of a Bacopa plant, a Centella plant, a Jatropha plant, an Aegle plant, a Terminalia plant, a Phyllanthus plant, a Spilanthes plant, or mixtures thereof; a skin/hair softening active extract of a Bacopa plant, a Centella plant, a Jatropha plant, an Aegle plant, a Terminalia plant, a Phyllanthus plant, a Spilanthes plant, or mixtures thereof; a skin/hair treatment active extract of a Bacopa plant, a Centella plant, a Jatropha plant, an Aegle plant, a Terminalia plant, a Phyllanthus plant, a Spilanthes plant, or mixtures thereof; an anti-inflammatory active extract of a Bacopa plant, a Centella plant, a Jatropha plant, an Aegle plant, a Terminalia plant, a Phyllanthus plant, a Spilanthes plant, or mixtures thereof; a cosmeceutical preservative active extract of a Bacopa plant, a Centella plant, a Jatropha plant, an Aegle plant, a Terminalia plant, a Phyllanthus plant, a Spilanthes plant, or mixtures thereof; a cosmeceutical stabilizer active extract of a Bacopa plant, a Centella plant, a Jatropha plant, an Aegle plant, a Terminalia plant, a Phyllanthus plant, a Spilanthes plant, or mixtures thereof; an antioxidant active extract of a Bacopa plant, a Centella plant, a Jatropha plant, an Aegle plant, a Terminalia plant, a Phyllanthus plant, a Spilanthes plant, or mixtures thereof; a free radical inhibitive extract of a Bacopa plant, a Centella plant, a Jatropha plant, an Aegle plant, a Terminalia plant, a Phyllanthus plant, a Spilanthes plant, or mixtures thereof; a cleansing active extract of a Bacopa plant, a Centella plant, a Jatropha plant, an Aegle plant, a Terminalia plant, a Phyllanthus plant, a Spilanthes plant, or mixtures thereof; an anti-microbial active extract of a Bacopa plant, a Centella plant, a Jatropha plant, an Aegle plant, a Terminalia plant, a Phyllanthus plant, a Spilanthes plant, or mixtures thereof; anti-fungal active extract of a Bacopa plant, a Centella plant, a Jatropha plant, an Aegle plant, a Terminalia plant, a Phyllanthus plant, a Spilanthes plant, or mixtures thereof; a hair coloring active extract of a Bacopa plant, a Centella plant, a Jatropha plant, an Aegle plant, a Terminalia plant, a Phyllanthus plant, a Spilanthes plant, or mixtures thereof; and an oral cleaning/deodoring active extract of a Bacopa plant, a Centella plant, a Jatropha plant, an Aegle plant, a Terminalia plant, a Phyllanthus plant, a Spilanthes plant, or mixtures thereof.

3. The extract of claim 1 which comprises at least one component selected from the group consisting of alkaloids, saponins, flavonoids, betulic acid, stigmastarol, β-sitosterol, bacosides, bacopasaponins, triterpene acids, triterpene glycosides, oils, lectins, cyclic triperpenes, tannins, lignans, pectins, and alkylamides.

4. The extract of claim 3 wherein said alkanoids are selected from the group consisting of brahmine and herpestine; said saponins are selected from the group consisting of d-mannitol, hersaponin, acid A and monnierin; said flavonoids are selected from the group consisting of luteolin and apigenin; said bacosides are selected from the group consisting of bacosides A, bacosides B, bacopaside II, bacopaside I, bacopaside X, bacopaside N2, bacopaside N1, bacopaside III, bacopaside IV, and bacopaside V; said bacopasaponins are selected from the group consisting of bacopasaponin C, bacopasaponin F, and bacopasaponin E; said triterpene acids are selected from the group consisting of asiatic acid, brahmic acid, isobrahmic acid, madecassic acid, and betulinic acid; and said triterpene glycosides are selected from the group consisting of asiaticoside, madecassoside, brahmoside, and brahminoside.

5. The extract of claim 1 which is obtained using an extraction medium comprising at least one solvent selected from the group consisting of water, low molecular weight alcohols, esters, hydrocarbons, ketones, halogen-containing hydrocarbons, and mixtures thereof.

6. The extract of claim 1 wherein the Bacopa plant is selected from the group consisting of Bacopa australis, Bacopa caroliniana, Bacopa crenata, Bacopa eisenii, Bacopa madagascariensis, Bacopa monniera, Bacopa myriophylloides, Bacopa rotundifolia, and mixtures thereof; the Centella plant is selected from the group consisting of Centella asiatica, Centella erecta, and mixtures thereof; the Jatropha plant is selected from the group consisting of Jatropha curcas, Jatropha gossypifolia, and mixtures thereof; the Aegle plant is selected from the group consisting of Aegle marmelos, and mixtures thereof; the Terminalia plant is selected from the group consisting of Terminalia chebula, Terminalia bellerica, and mixtures thereof; the Phyllanthus plant is selected from the group consisting of Phyllanthus emblica (Emblica officinalis), and mixtures thereof; and the Spilanthes plant is selected from the group consisting of Spilanthes acmella (Acmella oleracea), and mixtures thereof.

7. The extract of claim 1 wherein the Bacopa plant is Bacopa monniera, the Centella plant is Centella asiatica, the Jatropha plant is Jatropha curcas, the Aegle plant is Aegle marmelos, the Terminalia plant is Terminalia chebula or Terminalia bellerica, the Phyllanthus plant is Phyllanthus emblica (Emblica officinalis), and the Spilanthes plant is Spilanthes acmella (Acmella oleracea).

8. A cosmeceutical composition comprising, in an effective concentration, at least one of a Bacopa plant extract, a Centella plant extract, a Jatropha plant extract, an Aegle plant extract, a Terminalia plant extract, a Phyllanthus plant extract, a Spilanthes plant extract, or mixtures thereof; and at least one cosmeceutically acceptable carrier.

9. The cosmeceutical composition of claim 8 wherein said Bacopa plant extract is obtained from Bacopa australis, Bacopa caroliniana, Bacopa crenata, Bacopa eisenii, Bacopa madagascariensis, Bacopa monniera, Bacopa myriophylloides, Bacopa rotundifolia, or mixtures thereof; the Centella plant extract is obtained from Centella asiatica, Centella erecta, or mixtures thereof; the Jatropha plant is obtained from Jatropha curcas, Jatropha gossypifolia, or mixtures thereof; the Aegle plant extract is obtained from Aegle marmelos, or mixtures thereof; the Terminalia plant extract is obtained from Terminalia chebula, Terminalia bellerica, or mixtures thereof; the Phyllanthus plant extract is obtained from Phyllanthus emblica (Emblica officinalis), or mixtures thereof; and the Spilanthes plant is obtained from Spilanthes acmella (Acmella oleracea), or mixtures thereof.

10. The cosmeceutical composition of claim 8 wherein said Bacopa plant extract is obtained from Bacopa monniera, said Centella plant extract is obtained from Centella asiatica, said Jatropha plant extract is obtained from Jatropha curcas, said Aegle plant extract is obtained from Aegle marmelos; said Terminalia plant extract is obtained from Terminalia chebula or Terminalia bellerica; said Phyllanthus plant extract is obtained from Phyllanthus emblica (Emblica officinalis); and said Spilanthes plant extract is obtained from Spilanthes acmella (Acmella oleracea).

11. The cosmeceutical composition of claim 8 wherein said Bacopa plant extract comprises at least one component selected from the group consisting of alkaloids, saponins, flavonoids, betulic acid, stigmastarol, β-sitosterol, bacosides and bacopasaponins; said Centella plant extract comprises at least one component selected from the group consisting of triterpene acids and triterpene glycosides; said Jatropha plant extract comprises at least one component selected from the group consisting of oils, lectins and saponins; said Aegle plant extract comprises at least one component selected from the group consisting of marmalosin, coumarin, umbelliferone, d-limonene, sterols and triterpenoids; said Terminalia plant extract comprises at least one component selected from the group consisting of cyclic triterpenes and their derivatives, flavonoids, tannins, and other aromatics; said Phyllanthus plant extract comprises at least one component selected from the group consisting of lignans, tannins, and pectins; and said Spilanthes plant extract comprises at least one component selected from the group consisting of alkylamides, triterpenes, spilanthol, and stigmasterol.

12. The cosmeceutical composition of claim 11 wherein said alkanoids are selected from the group consisting of brahmine and herpestine; said saponins are selected from the group consisting of d-mannitol, hersaponin, acid A and monnierin; said flavonoids are selected from the group consisting of luteolin and apigenin; said bacosides are selected from the group consisting of bacosides A, bacosides B, bacopaside II, bacopaside I, bacopaside X, bacopaside N2, bacopaside N1, bacopaside III, bacopaside IV, and bacopaside V; said bacopasaponins are selected from the group consisting of bacopasaponin C, bacopasaponin F, and bacopasaponin E; said triterpene acids are selected from the group consisting of asiatic acid, brahmic acid, isobrahmic acid, madecassic acid, and betulinic acid; and said triterpene glycosides are selected from the group consisting of asiaticoside, madecassoside, brahmoside, and brahminoside.

13. The cosmeceutical composition of claim 8 wherein said Bacopa plant extract is obtained from any part of a Bacopa plant, said Centella plant extract is obtained from any part of a Centella plant, said Jatropha plant extract is obtained from any part of a Jatropha plant, said Aegle plant extract is obtained from any part of an Aegle plant, said Terminalia plant extract is obtained from any part of an Terminalia plant, said Phyllanthus plant extract is obtained from any part of a Phyllanthus plant; and said Spilanthes plant extract is obtained from any part of a Spilanthes plant.

14. The cosmeceutical composition of claim 8 wherein said Bacopa plant extract, Centella plant extract, Jatropha plant extract, Aegle plant extract, Terminalia plant extract, Phyllanthus plant extract, Spilanthes plant extract, or mixtures thereof, is about 0.001 weight percent to about 25 weight percent of the total weight of the cosmeceutical composition.

15. The cosmeceutical composition of claim 8 wherein said cosmeceutically acceptable carrier is selected from the group consisting of water, liquid or solid emollients, solvents, humectants, thickeners, powders, and perfumes.

16. The cosmeceutical composition of claim 8 wherein said cosmeceutically acceptable carrier is about 0.001 weight percent to about 99.9 weight percent of the total weight of the cosmeceutical composition.

17. The cosmeceutical composition of claim 8 which is used in topical form.

18. The cosmeceutical composition of claim 17 in which the topical form is selected from a solution, emulsion, serum, skin and/or hair cleanser, body wash, body scrub, bar soap, liquid soap, shampoo, lather, deodorant, skin and/or hair care preparation, foam, mousse, cream, lotion, pomade, balm, stick, gel, pump spray, aerosol spray, and combinations thereof.

19. The cosmeceutical composition of claim 8 which is selected from a sunless tanning composition, a sunscreen composition, an ultraviolet radiation protective composition, an anti-aging composition, an anti-wrinkle composition, a moisturizer composition, a skin soothing composition, a skin/hair softening composition, a skin/hair treating composition, an anti-inflammatory composition, a cosmeceutical preservative composition, a cosmeceutical stabilizer composition, an antioxidant composition, a free radical inhibitive composition, a cleansing composition, an anti-microbial composition, an anti-fungal composition, a hair coloring composition, and an oral cleaning/deodoring composition.

20. A method of protecting or treating skin and/or hair of humans which comprises applying to the skin and/or hair a cosmeceutical composition comprising, in an effective concentration, at least one of a Bacopa plant extract, a Centella plant extract, a Jatropha plant extract, an Aegle plant extract, a Terminalia plant extract, a Phyllanthus plant extract, a Spilanthes plant extract, or mixtures thereof, and at least one cosmeceutically acceptable carrier.

21. The method of claim 20 wherein the cosmeceutical composition is selected from a sunless tanning composition, a sunscreen composition, an ultraviolet radiation protective composition, an anti-aging composition, an anti-wrinkle composition, a moisturizer composition, a skin soothing composition, a skin/hair softening composition, a skin/hair treating composition, an anti-inflammatory composition, a cosmeceutical preservative composition, a cosmeceutical stabilizer composition, an antioxidant composition, a free radical inhibitive composition, a cleansing composition, an anti-microbial composition, an anti-fungal composition, a hair coloring composition, and an oral cleaning/deodoring composition.

22. A process for preparing an extract, said process comprising: (i) providing a Bacopa plant, a Centella plant, a Jatropha plant, an Aegle plant, a Terminalia plant, a Phyllanthus plant, a Spilanthes plant, or mixtures thereof; (ii) providing an extraction medium comprising at least one solvent selected from the group consisting of water, low molecular weight alcohols, esters, hydrocarbons, ketones, halogen-containing hydrocarbons, and mixtures thereof; and (iii) contacting the Bacopa plant, Centella plant, Jatropha plant, Aegle plant, Terminalia plant, Phyllanthus plant, Spilanthes plant, or mixtures thereof with the extraction medium.

23. An extract selected from the group consisting of a Bacopa plant extract, a Centella plant extract, a Jatropha plant extract, an Aegle plant extract, a Terminalia plant extract, a Phyllanthus plant extract, a Spilanthes plant extract, and mixtures thereof.

24. An extract selected from the group consisting of a stabilizer active extract of a Bacopa plant, a Centella plant, a Jatropha plant, an Aegle plant, a Terminalia plant, a Phyllanthus plant, a Spilanthes plant, or mixtures thereof; a surfactant active extract a Bacopa plant, a Centella plant, a Jatropha plant, an Aegle plant, a Terminalia plant, a Phyllanthus plant, a Spilanthes plant, or mixtures thereof; a preservative active extract a Bacopa plant, a Centella plant, a Jatropha plant, an Aegle plant, a Terminalia plant, a Phyllanthus plant, a Spilanthes plant, or mixtures thereof; a treatment active extract a Bacopa plant, a Centella plant, a Jatropha plant, an Aegle plant, a Terminalia plant, a Phyllanthus plant, a Spilanthes plant, or mixtures thereof; a dispersant active extract of a Bacopa plant, a Centella plant, a Jatropha plant, an Aegle plant, a Terminalia plant, a Phyllanthus plant, a Spilanthes plant, or mixtures thereof, a chelating active extract of a Bacopa plant, a Centella plant, a Jatropha plant, an Aegle plant, a Terminalia plant, a Phyllanthus plant, a Spilanthes plant, or mixtures thereof; an emulsifier active extract of a Bacopa plant, a Centella plant, a Jatropha plant, an Aegle plant, a Terminalia plant, a Phyllanthus plant, a Spilanthes plant, or mixtures thereof; a surface modifying active extract of a Bacopa plant, a Centella plant, a Jatropha plant, an Aegle plant, a Terminalia plant, a Phyllanthus plant, a Spilanthes plant, or mixtures thereof; a foaming/frothing active extract of a Bacopa plant, a Centella plant, a Jatropha plant, an Aegle plant, a Terminalia plant, a Phyllanthus plant, a Spilanthes plant, or mixtures thereof; a surface active agent active extract of a Bacopa plant, a Centella plant, a Jatropha plant, an Aegle plant, a Terminalia plant, a Phyllanthus plant, a Spilanthes plant, or mixtures thereof; a cleansing/degreasing active extract of a Bacopa plant, a Centella plant, a Jatropha plant, an Aegle plant, a Terminalia plant, a Phyllanthus plant, a Spilanthes plant, or mixtures thereof; and a rheology modifying active extract of a Bacopa plant, a Centella plant, a Jatropha plant, an Aegle plant, a Terminalia plant, a Phyllanthus plant, a Spilanthes plant, or mixtures thereof.

25. The extract of claim 23 wherein the Bacopa plant is selected from the group consisting of Bacopa australis, Bacopa caroliniana, Bacopa crenata, Bacopa eisenii, Bacopa madagascariensis, Bacopa monniera, Bacopa myriophylloides, Bacopa rotundifolia, and mixtures thereof; the Centella plant is selected from the group consisting of Centella asiatica and Centella erecta, and mixtures thereof; the Jatropha plant is selected from Jatropha curcas, Jatropha gossypifolia, and mixtures thereof; the Aegle plant is selected from Aegle marmelos, and mixtures thereof; the Terminalia plant is selected from Terminalia chebula, Terminalia bellerica, and mixtures thereof; the Phyllanthus plant is selected from Phyllanthus emblica (Emblica officinalis), and mixtures thereof; and the Spilanthes plant is selected from Spilanthes acmella (Acmella oleracea), and mixtures thereof.

26. The extract of claim 23 wherein the Bacopa plant is Bacopa monniera, the Centella plant is Centella asiatica, the Jatropha plant is Jatropha curcas, the Aegle plant is Aegle marmelos, the Terminalia plant is Terminalia chebula or Terminalia bellerica, the Phyllanthus plant is Phyllanthus emblica (Emblica officinalis), and the Spilanthes plant is Spilanthes acmella (Acmella oleracea).

27. A composition comprising at least one of a Bacopa plant extract, a Centella plant extract, a Jatropha plant extract, an Aegle plant extract, a Terminalia plant extract, a Phyllanthus plant extract, a Spilanthes plant extract, or mixtures thereof.

28. The composition of claim 27 wherein said Bacopa plant extract is obtained from Bacopa australis, Bacopa caroliniana, Bacopa crenata, Bacopa eisenii, Bacopa madagascariensis, Bacopa monniera, Bacopa myriophylloides, Bacopa rotundifolia, and mixtures thereof; said Centella plant extract is obtained from Centella asiatica and Centella erecta, and mixtures thereof; said Jatropha plant extract is obtained from Jatropha curcas, Jatropha gossypifolia, or mixtures thereof; said Aegle plant extract is obtained from any part of an Aegle plant, and mixtures thereof; said Terminalia plant extract is obtained from any part of an Terminalia plant, and mixtures thereof; said Phyllanthus plant extract is obtained from any part of a Phyllanthus plant, and mixtures thereof; and said Spilanthes plant extract is obtained from any part of a Spilanthes plant, and mixtures thereof.

29. The composition of claim 27 wherein said Bacopa plant extract is obtained from Bacopa monniera, said Centella plant extract is obtained from Centella asiatica, said Jatropha plant extract is obtained from Jatropha curcas; said Aegle plant extract is obtained from Aegle marmelos; said Terminalia plant extract is obtained from Terminalia chebula or Terminalia bellerica; said Phyllanthus plant extract is obtained from Phyllanthus emblica (Emblica officinalis); and said Spilanthes plant extract is obtained from Spilanthes acmella (Acmella oleracea).

30. The composition of claim 27 which is selected from a stabilizer composition, a surfactant composition, a preservative composition, a treatment composition, a dispersant composition, a chelating composition, an emulsifier composition, a surface modifying composition, a foaming/frothing composition, a surface active agent composition, a cleansing/degreasing composition, and a rheology modifying composition.

31. The composition of claim 30 wherein the stabilizer composition is used in coatings, paints, polymers, plastics and emulsions; the surfactant composition is used in oil recovery, soil cleaning, mineral separation, agrochemicals, detergents, food product preparation, nutraceuticals, coating, printing inks, electronic printing, microelectronics, magnetic recording, high technology electronic ceramics, batteries, and particle dispersion; the preservative composition is used in foods; the treatment composition is used in fabric and fiber care; the dispersant composition is used in oil recovery, oil cleanup, food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, biotechnology, particle dispersion, laundry, detergents, cleaning, washing (e.g., automobiles, trucks, trains, airplanes, and the like), and degreasing; the chelating composition is used in metal extraction and washing (e.g., automobiles, trucks, trains, airplanes, and the like); the emulsifier composition is used in oil recovery, oil cleanup, food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, and biotechnology; the surface modifying composition is used in food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, electronic printing, microelectronics, magnetic recording, high technology electronic ceramics, biotechnology, separation techniques, and particle dispersion; the foaming/frothing composition is used in fire fighting and oil recovery; the surface active agent composition is used in oil recovery, soil cleaning, mineral separation, agrochemicals, detergents, food product preparation, nutraceuticals, coating, printing inks, electronic printing, microelectronics, magnetic recording, high technology electronic ceramics, batteries, and particle dispersion; the cleansing/degreasing composition is used in food product preparation, nutraceuticals, pharmaceuticals, cosmetics, agrochemicals, paints, paper making, printing and graphic arts, lithography, hydraulic fluid emulsion, fuels, lubricants, coatings and polymers, bioactive materials, extraction media, electronic printing, microelectronics, magnetic recording, high technology electronic ceramics, biotechnology, laundry, detergents, and washing (e.g., automobiles, trucks, trains, airplanes, and the like); and the rheology modifying composition is used in thickening and thinning of fluids, suspensions, polymers, foods and additives, bodily fluids (e.g., blood) and other biological materials.

32. A method comprising: (i) providing a substrate or medium; (ii) providing a composition comprising at least one of a Bacopa plant extract, a Centella plant extract, a Jatropha plant extract, an Aegle plant extract, a Terminalia plant extract, a Phyllanthus plant extract, a Spilanthes plant extract, or mixtures thereof; and (iii) contacting the substrate or medium with the composition.

33. The method of claim 32 wherein the composition is selected from a stabilizer composition, a surfactant composition, a preservative composition, a treatment composition, a dispersant composition, a chelating composition, an emulsifier composition, a surface modifying composition, a foaming/frothing composition, a surface active agent composition; a cleansing/degreasing composition; and a rheology modifying composition.
